SEQUENCE LISTING (172 sequences)

SEQ ID NO: 1 - protein, 98 aa, Homo sapiens
  Seq ID No. 1 VH-H-3
EVQLEQSGGGLVQPGGSLRLSCAASGFIFSSYGMTWVRQAPGKGLEWVSAISGSGGSTYYADSVKGRFTISRDNSKNTLYLQMNSLRAEDTAVYYCAR

SEQ ID NO: 2 - protein, 98 aa, Homo sapiens
  Seq ID No. 2 VH3-93
QVQLQESGGGLVQPGGSLRLSCAASGFTFSSYAVSWVRQAPGKGLEWVSAISGSGDRTYYADSVRGRFTISRDNSKNTLYLQMNSLRAEDTAVYYCAR

SEQ ID NO: 3 - protein, 98 aa, Homo sapiens
  Seq ID No. 3 81G1
QVQLQESGGGLVQPGGSLRLSCAASGFTLSNYAMSWVRQAPGKGLEWVSTIRGSDGTTFYSDSVRGRFTISRDNSKNTLYLQMDSLRAEDTAVYYCAR

SEQ ID NO: 4 - DNA, 294 bp, Homo sapiens
  source 1..294: /organism=“Homo sapiens” /note=“Seq ID No. 4 VH-H-3 ” /mol_type=“unassigned DNA”
gaggtgcagctggagcagtctggaggaggcttggtccagcctggggggtccctgagactctcctgtgcagcctctggattcatctttagcagctatggcatgacctgggtccgccaggctccagggaaggggctggagtgggtctcagctatcagtggtagtggtggtagcacatactacgcagactccgtgaagggccgattcaccatctccagagacaattccaagaacacgctgtatctgcaaatgaacagcctgagagccgaggacacggccgtgtattactgtgcgaga

SEQ ID NO: 5 - DNA, 294 bp, Homo sapiens
  source 1..294: /organism=“Homo sapiens” /note=“Seq ID No. 5 VH3-93 ” /mol_type=“unassigned DNA”
caggtgcagctccaggagtctggaggaggcttggtacagcctggggggtccctgagactctcctgtgcggcctctggattcacctttagcagctatgccgtgagctgggtccgccaggctccagggaaggggctggagtgggtctcagctattagtggtagtggtgataggacatactacgcagactccgtgaggggccggttcaccatctccagagacaattccaagaacacgctgtatctgcaaatgaacagcctgagagccgaggacacggccgtgtattactgtgcaaga

SEQ ID NO: 6 - DNA, 295 bp, Homo sapiens
  source 1..295: /organism=“Homo sapiens” /note=“Seq ID No. 6 81G1 ” /mol_type=“unassigned DNA”
caggtgcagctccaggagtctgggggaggcttggtacagcctggggggtccctgagactctcctgtgcagcttccgggttcacccttagcaactatgccatgagctgggtccgccaggctccagggaaggggctggagtgggtctcaactattcgtggcagtgatggtaccacattctactcagactctgtgaggggccgattcaccatctccagagacaattccaagaacacgctgtatctgcaaatggacagcctgagagccgaggacacggccgtgtattactgtgcgagag

SEQ ID NO: 7 - DNA, 62 bp, Artificial Sequence
  source 1..62: /organism=“Artificial Sequence” /note=“PCR Primer” /mol_type=“unassigned DNA”
  misc_feature 45: /note=“n = a or g or c or t/u, unknown, or other”
  misc_feature 40: /note=“s = g or c”
  misc_feature 42: /note=“r = g or a”
  misc_feature 46: /note=“s = g or c”
  misc_feature 48: /note=“r = g or a”
  misc_feature 51: /note=“b = g or c or t/u”
  misc_feature 53: /note=“w = a or t/u”
  misc_feature 54: /note=“r = g or a”
  misc_feature 55: /note=“s = g or c”
  misc_feature 60: /note=“y = t/u or c”
gcagctaatacgactcactatagggaacagaccaccatgsargtnsarctbgwrsagtcygg

SEQ ID NO: 8 - DNA, 42 bp, Artificial Sequence
  source 1..42: /organism=“Artificial Sequence” /note=“PCR Primer” /mol_type=“unassigned DNA”
gctaccgccaccctcgagtgaagagacggtgaccagtgtccc

SEQ ID NO: 9 - DNA, 39 bp, Artificial Sequence
  source 1..39: /organism=“Artificial Sequence” /note=“PCR Primer” /mol_type=“unassigned DNA”
ctcgagggtggcggtagcactgtggctgcaccatctgtc

SEQ ID NO: 10 - DNA, 21 bp, Artificial Sequence
  source 1..21: /organism=“Artificial Sequence” /note=“PCR Primer” /mol_type=“unassigned DNA”
gcactctcccctgttgaagct

SEQ ID NO: 11 - DNA, 72 bp, Artificial Sequence
  source 1..72: /organism=“Artificial Sequence” /note=“PCR Primer” /mol_type=“unassigned DNA”
gcactctcccctgttgaagctctttgtgacgggcgagctcaggccctgatgggtgacttcgcaggcgtagac

SEQ ID NO: 12 - DNA, 38 bp, Artificial Sequence
  source 1..38: /organism=“Artificial Sequence” /note=“PCR Primer” /mol_type=“unassigned DNA”
gcagctaatacgactcactataggaacagaccaccatg

SEQ ID NO: 13 - DNA, 32 bp, Artificial Sequence
  source 1..32: /organism=“Artificial Sequence” /note=“PCR Primer” /mol_type=“unassigned DNA”
gaacagaccaccatgacttcgcaggcgtagac

SEQ ID NO: 14 - DNA, 15 bp, Artificial Sequence
  source 1..15: /organism=“Artificial Sequence” /note=“PCR Primer” /mol_type=“unassigned DNA”
gaacagaccaccatg

SEQ ID NO: 15 - DNA, 15 bp, Artificial Sequence
  source 1..15: /organism=“Artificial Sequence” /note=“PCR Primer” /mol_type=“unassigned DNA”
gatctcgatcccgcg

SEQ ID NO: 16 - DNA, 23 bp, Artificial Sequence
  source 1..23: /organism=“Artificial Sequence” /note=“PCR Primer” /mol_type=“unassigned DNA”
catggtatatctccttcttaaag

SEQ ID NO: 17 - DNA, 36 bp, Artificial Sequence
  source 1..36: /organism=“Artificial Sequence” /note=“PCR Primer” /mol_type=“unassigned DNA”
ctcgagggtggcggtagccaccaccaccaccaccac

SEQ ID NO: 18 - DNA, 18 bp, Artificial Sequence
  source 1..18: /organism=“Artificial Sequence” /note=“PCR Primer” /mol_type=“unassigned DNA”
tccggatatagttcctcc

SEQ ID NO: 19 - DNA, 46 bp, Artificial Sequence
  source 1..46: /organism=“Artificial Sequence” /note=“PCR Primer” /mol_type=“unassigned DNA”
  misc_feature 29: /note=“n = a or g or c or t/u, unknown, or other”
  misc_feature 24: /note=“s = g or c”
  misc_feature 26: /note=“r = g or a”
  misc_feature 30: /note=“s = g or c”
  misc_feature 32: /note=“r = g or a”
  misc_feature 35: /note=“b = g or c or t/u”
  misc_feature 37: /note=“w = a or t/u”
  misc_feature 38: /note=“r = g or a”
  misc_feature 39: /note=“s = g or c”
  misc_feature 44: /note=“y = t/u or c”
ctttaagaaggagatataccatgsargtnsarctbgwrsagtcygg

SEQ ID NO: 20 - DNA, 64 bp, Artificial Sequence
  source 1..64: /organism=“Artificial Sequence” /note=“PCR Primer” /mol_type=“unassigned DNA”
gcagctaatacgactcactataggaacagaccaccatggacgaggtgcagctggagcagtctgg

SEQ ID NO: 21 - DNA, 42 bp, Artificial Sequence
  source 1..42: /organism=“Artificial Sequence” /note=“PCR Primer” /mol_type=“unassigned DNA”
ggaacagaccaccatggcccaggtgcagctccaggagtctgg

SEQ ID NO: 22 - DNA, 24 bp, Artificial Sequence
  source 1..24: /organism=“Artificial Sequence” /note=“PCR Primer” /mol_type=“unassigned DNA”
ggacacggccgtgtattactgtgc

SEQ ID NO: 23 - DNA, 34 bp, Artificial Sequence
  source 1..34: /organism=“Artificial Sequence” /note=“PCR Primer” /mol_type=“unassigned DNA”
  misc_feature 22: /note=“r = g or a”
  misc_feature 28: /note=“r = g or a”
gctaccgccaccctcgagtgargagacrgtgacc

SEQ ID NO: 24 - DNA, 30 bp, Artificial Sequence
  source 1..30: /organism=“Artificial Sequence” /note=“PCR Primer” /mol_type=“unassigned DNA”
gtccatggccatcgccggctgggccgcgag

SEQ ID NO: 25 - DNA, 49 bp, Artificial Sequence
  source 1..49: /organism=“Artificial Sequence” /note=“PCR Primer” /mol_type=“unassigned DNA”
tagcagcctcgagggtggcggtagccatcaccaccatcaccacgggagc

SEQ ID NO: 26 - protein, 9 aa, Homo sapiens
  CDR3 sequence
PAGYDAFDI

SEQ ID NO: 27 - protein, 12 aa, Homo sapiens
  CDR3 sequence
DRGSSISDPFDI

SEQ ID NO: 28 - protein, 13 aa, Homo sapiens
  CDR3 sequence
EAPWLAQYDAFDI

SEQ ID NO: 29 - protein, 10 aa, Homo sapiens
  CDR3 sequence
GQDGYDGFDI

SEQ ID NO: 30 - protein, 10 aa, Homo sapiens
  CDR3 sequence
PSELSGWFSP

SEQ ID NO: 31 - protein, 11 aa, Homo sapiens
  CDR3 sequence
DKWDDIKQFDN

SEQ ID NO: 32 - protein, 14 aa, Homo sapiens
  CDR3 sequence
DSDVDMYGYYTFES

SEQ ID NO: 33 - protein, 15 aa, Homo sapiens
  CDR3 sequence
EASYYDTTGYKIFDL

SEQ ID NO: 34 - protein, 14 aa, Homo sapiens
  CDR3 sequence
EMDYDKVGYSQFDY

SEQ ID NO: 35 - protein, 15 aa, Homo sapiens
  CDR3 sequence
EPGRYYFDGSDYEDV

SEQ ID NO: 36 - protein, 13 aa, Homo sapiens
  CDR3 sequence
ESPYNDDHYIMDS

SEQ ID NO: 37 - protein, 13 aa, Homo sapiens
  CDR3 sequence
EVEYGGGLYDFDV

SEQ ID NO: 38 - protein, 7 aa, Homo sapiens
  CDR3 sequence
KWNDVDS

SEQ ID NO: 39 - protein, 8 aa, Homo sapiens
  CDR3 sequence
QWNNWHPN

SEQ ID NO: 40 - protein, 4 aa, Homo sapiens
  CDR3 sequence
DAQI

SEQ ID NO: 41 - protein, 4 aa, Homo sapiens
  CDR3 sequence
DEDI

SEQ ID NO: 42 - protein, 4 aa, Homo sapiens
  CDR3 sequence
DEDT

SEQ ID NO: 43 - protein, 9 aa, Homo sapiens
  CDR3 sequence
DEPPGAFDI

SEQ ID NO: 44 - protein, 12 aa, Homo sapiens
  CDR3 sequence
DGAAAGLDAFDI

SEQ ID NO: 45 - protein, 4 aa, Homo sapiens
  CDR3 sequence
DKDI

SEQ ID NO: 46 - protein, 4 aa, Homo sapiens
  CDR3 sequence
DKDY

SEQ ID NO: 47 - protein, 4 aa, Homo sapiens
  CDR3 sequence
DKHI

SEQ ID NO: 48 - protein, 4 aa, Homo sapiens
  CDR3 sequence
DMQQ

SEQ ID NO: 49 - protein, 7 aa, Homo sapiens
  CDR3 sequence
DNMAFDI

SEQ ID NO: 50 - protein, 4 aa, Homo sapiens
  CDR3 sequence
DQDY

SEQ ID NO: 51 - protein, 9 aa, Homo sapiens
  CDR3 sequence
DSSGWPFDY

SEQ ID NO: 52 - protein, 10 aa, Homo sapiens
  CDR3 sequence
EDGTIGAFDI

SEQ ID NO: 53 - protein, 10 aa, Homo sapiens
  CDR3 sequence
EDLESSGEDS

SEQ ID NO: 54 - protein, 9 aa, Homo sapiens
  CDR3 sequence
EDYGDAFDI

SEQ ID NO: 55 - protein, 11 aa, Homo sapiens
  CDR3 sequence
EGSGSRYAFDI

SEQ ID NO: 56 - protein, 9 aa, Homo sapiens
  CDR3 sequence
EGYGDAFDI

SEQ ID NO: 57 - protein, 4 aa, Homo sapiens
  CDR3 sequence
EIGI

SEQ ID NO: 58 - protein, 8 aa, Homo sapiens
  CDR3 sequence
EIQTGDDY

SEQ ID NO: 59 - protein, 8 aa, Homo sapiens
  CDR3 sequence
EKDYGMDV

SEQ ID NO: 60 - protein, 8 aa, Homo sapiens
  CDR3 sequence
ELAGAFDI

SEQ ID NO: 61 - protein, 8 aa, Homo sapiens
  CDR3 sequence
ENRDGEDV

SEQ ID NO: 62 - protein, 8 aa, Homo sapiens
  CDR3 sequence
ENSYDTDV

SEQ ID NO: 63 - protein, 8 aa, Homo sapiens
  CDR3 sequence
ETQTGDDY

SEQ ID NO: 64 - protein, 12 aa, Homo sapiens
  CDR3 sequence
EWPLAGPDAFDI

SEQ ID NO: 65 - protein, 8 aa, Homo sapiens
  CDR3 sequence
EYDYGMDV

SEQ ID NO: 66 - protein, 8 aa, Homo sapiens
  CDR3 sequence
EYDYGTDV

SEQ ID NO: 67 - protein, 8 aa, Homo sapiens
  CDR3 sequence
EYHYGEDV

SEQ ID NO: 68 - protein, 13 aa, Homo sapiens
  CDR3 sequence
FIRGNWLPDAFDL

SEQ ID NO: 69 - protein, 9 aa, Homo sapiens
  CDR3 sequence
GPSHGGFDI

SEQ ID NO: 70 - protein, 10 aa, Homo sapiens
  CDR3 sequence
GRRGWSAFDI

SEQ ID NO: 71 - protein, 4 aa, Homo sapiens
  CDR3 sequence
NEDV

SEQ ID NO: 72 - protein, 13 aa, Homo sapiens
  CDR3 sequence
SFYIEGRTRAFGI

SEQ ID NO: 73 - protein, 4 aa, Homo sapiens
  CDR3 sequence
DKDN

SEQ ID NO: 74 - protein, 4 aa, Homo sapiens
  CDR3 sequence
DKDV

SEQ ID NO: 75 - protein, 16 aa, Homo sapiens
  CDR3 sequence
EARGGGYSMGYGSFDY

SEQ ID NO: 76 - protein, 12 aa, Homo sapiens
  CDR3 sequence
EDDFQNSYYVDV

SEQ ID NO: 77 - protein, 12 aa, Homo sapiens
  CDR3 sequence
EDNFEDSYYVDV

SEQ ID NO: 78 - protein, 11 aa, Homo sapiens
  CDR3 sequence
EDWNLGRGMDV

SEQ ID NO: 79 - protein, 14 aa, Homo sapiens
  CDR3 sequence
EDYGDSQYLEALDV

SEQ ID NO: 80 - protein, 13 aa, Homo sapiens
  CDR3 sequence
EPYDDYDSDSMDV

SEQ ID NO: 81 - protein, 12 aa, Homo sapiens
  CDR3 sequence
ERPGREFYGMDV

SEQ ID NO: 82 - protein, 7 aa, Homo sapiens
  CDR3 sequence
ESDMGDV

SEQ ID NO: 83 - protein, 11 aa, Homo sapiens
  CDR3 sequence
GKTAAAGGFDN

SEQ ID NO: 84 - protein, 11 aa, Homo sapiens
  CDR3 sequence
GLYRHGQGLDP

SEQ ID NO: 85 - protein, 13 aa, Homo sapiens
  CDR3 sequence
GMYNWNDRNALDI

SEQ ID NO: 86 - protein, 13 aa, Homo sapiens
  CDR3 sequence
GPHDSSYYYGLDV

SEQ ID NO: 87 - protein, 8 aa, Homo sapiens
  CDR3 sequence
GTQRQLSP

SEQ ID NO: 88 - protein, 11 aa, Homo sapiens
  CDR3 sequence
GYFDWLAPPVV

SEQ ID NO: 89 - protein, 14 aa, Homo sapiens
  CDR3 sequence
LHHDFWSVDDTFDV

SEQ ID NO: 90 - protein, 15 aa, Homo sapiens
  CDR3 sequence
QNCGSPDCSYGGFDP

SEQ ID NO: 91 - protein, 16 aa, Homo sapiens
  CDR3 sequence
RFFDWLQGSRYYGMDV

SEQ ID NO: 92 - protein, 13 aa, Homo sapiens
  CDR3 sequence
AARGTRELSTVDV

SEQ ID NO: 93 - protein, 16 aa, Homo sapiens
  CDR3 sequence
AQTSGIYTYYYHTMDV

SEQ ID NO: 94 - protein, 12 aa, Homo sapiens
  CDR3 sequence
ASVGSRPHTFDI

SEQ ID NO: 95 - protein, 18 aa, Homo sapiens
  CDR3 sequence
DAGFGTGLSLRYYHYMDV

SEQ ID NO: 96 - protein, 10 aa, Homo sapiens
  CDR3 sequence
DDILTGRMDV

SEQ ID NO: 97 - protein, 12 aa, Homo sapiens
  CDR3 sequence
DFGDYGHSGFDM

SEQ ID NO: 98 - protein, 14 aa, Homo sapiens
  CDR3 sequence
DGGSGSLMHDAFDI

SEQ ID NO: 99 - protein, 13 aa, Homo sapiens
  CDR3 sequence
DHGDYYYYHSMDV

SEQ ID NO: 100 - protein, 19 aa, Homo sapiens
  CDR3 sequence
DIRLPASMRDDFFYFGMDV

SEQ ID NO: 101 - protein, 16 aa, Homo sapiens
  CDR3 sequence
DLFDLWSGYFHDAFDI

SEQ ID NO: 102 - protein, 17 aa, Homo sapiens
  CDR3 sequence
DLGHDFWSGYYHDAFDI

SEQ ID NO: 103 - protein, 12 aa, Homo sapiens
  CDR3 sequence
DPRKVAPRAFDI

SEQ ID NO: 104 - protein, 14 aa, Homo sapiens
  CDR3 sequence
DPVAGTSVPSGFDL

SEQ ID NO: 105 - protein, 17 aa, Homo sapiens
  CDR3 sequence
DPYSGRYGNEHYHYMDV

SEQ ID NO: 106 - protein, 17 aa, Homo sapiens
  CDR3 sequence
DPYSGRYGNEYYHYMDV

SEQ ID NO: 107 - protein, 17 aa, Homo sapiens
  CDR3 sequence
DRFLQRTWSRPHDAFDM

SEQ ID NO: 108 - protein, 9 aa, Homo sapiens
  CDR3 sequence
DSGYNAFDI

SEQ ID NO: 109 - protein, 16 aa, Homo sapiens
  CDR3 sequence
DSRGGGSYPYYHGMDV

SEQ ID NO: 110 - protein, 11 aa, Homo sapiens
  CDR3 sequence
DVYSSGRSFDY

SEQ ID NO: 111 - protein, 21 aa, Homo sapiens
  CDR3 sequence
DWGSHYCDSMGPRRPRKAFDI

SEQ ID NO: 112 - protein, 21 aa, Homo sapiens
  CDR3 sequence
DWGSYYHDSSDPRRPHEAFDL

SEQ ID NO: 113 - protein, 21 aa, Homo sapiens
  CDR3 sequence
DWGSYYHDSSGPRRPHEAFDI

SEQ ID NO: 114 - protein, 21 aa, Homo sapiens
  CDR3 sequence
DWGSYYYDSSGPRRPHEAFDI

SEQ ID NO: 115 - protein, 17 aa, Homo sapiens
  CDR3 sequence
EGQYLWLPRHYYHGLDV

SEQ ID NO: 116 - protein, 12 aa, Homo sapiens
  CDR3 sequence
EWVLGDKSVFDV

SEQ ID NO: 117 - protein, 12 aa, Homo sapiens
  CDR3 sequence
EYCRSETCLMDV

SEQ ID NO: 118 - protein, 18 aa, Homo sapiens
  CDR3 sequence
GAGYCSGGSCYPGGVFDI

SEQ ID NO: 119 - protein, 14 aa, Homo sapiens
  CDR3 sequence
GDFWSGAWHDAFDI

SEQ ID NO: 120 - protein, 18 aa, Homo sapiens
  CDR3 sequence
GDGYCSGGSCYPGGAFDI

SEQ ID NO: 121 - protein, 13 aa, Homo sapiens
  CDR3 sequence
GFWSGYLHDAFDI

SEQ ID NO: 122 - protein, 16 aa, Homo sapiens
  CDR3 sequence
GGSGHGSYYYFHTMDV

SEQ ID NO: 123 - protein, 13 aa, Homo sapiens
  CDR3 sequence
GGSGWYLSNAFDI

SEQ ID NO: 124 - protein, 16 aa, Homo sapiens
  CDR3 sequence
GGYCSSTSCLVHTFDI

SEQ ID NO: 125 - protein, 18 aa, Homo sapiens
  CDR3 sequence
GIAAVTKDYNYYYHAMDV

SEQ ID NO: 126 - protein, 18 aa, Homo sapiens
  CDR3 sequence
GIATVTKDYNYYYHAMDV

SEQ ID NO: 127 - protein, 14 aa, Homo sapiens
  CDR3 sequence
GISATDYYYHGMDV

SEQ ID NO: 128 - protein, 13 aa, Homo sapiens
  CDR3 sequence
GLERGDVFHHFDY

SEQ ID NO: 129 - protein, 14 aa, Homo sapiens
  CDR3 sequence
GLIDGDYYYHGMDV

SEQ ID NO: 130 - protein, 10 aa, Homo sapiens
  CDR3 sequence
GLPTDRAFDV

SEQ ID NO: 131 - protein, 15 aa, Homo sapiens
  CDR3 sequence
GLSGPQWHYYHYMDV

SEQ ID NO: 132 - protein, 15 aa, Homo sapiens
  CDR3 sequence
GPDYGGNGPVGAFDI

SEQ ID NO: 133 - protein, 14 aa, Homo sapiens
  CDR3 sequence
GPEGSSSFLGAFDI

SEQ ID NO: 134 - protein, 14 aa, Homo sapiens
  CDR3 sequence
GRIRDGYFHDAFDI

SEQ ID NO: 135 - protein, 12 aa, Homo sapiens
  CDR3 sequence
GSGRYYYHGMDV

SEQ ID NO: 136 - protein, 9 aa, Homo sapiens
  CDR3 sequence
GSGSWAFDI

SEQ ID NO: 137 - protein, 12 aa, Homo sapiens
  CDR3 sequence
GSVGTRPHTFDI

SEQ ID NO: 138 - protein, 12 aa, Homo sapiens
  CDR3 sequence
GSVGTRPHTFDV

SEQ ID NO: 139 - protein, 12 aa, Homo sapiens
  CDR3 sequence
GTAHSYYHLMDV

SEQ ID NO: 140 - protein, 11 aa, Homo sapiens
  CDR3 sequence
GTEYYYHDMDV

SEQ ID NO: 141 - protein, 14 aa, Homo sapiens
  CDR3 sequence
GTLVPTGHYHTLDV

SEQ ID NO: 142 - protein, 12 aa, Homo sapiens
  CDR3 sequence
GVAYSYYHHMDV

SEQ ID NO: 143 - protein, 12 aa, Homo sapiens
  CDR3 sequence
GVTSAFVFAFDI

SEQ ID NO: 144 - protein, 12 aa, Homo sapiens
  CDR3 sequence
GVTSAFVFAFDV

SEQ ID NO: 145 - protein, 12 aa, Homo sapiens
  CDR3 sequence
GVVGSRPHTFDI

SEQ ID NO: 146 - protein, 14 aa, Homo sapiens
  CDR3 sequence
GVVPAGHYYHYMDV

SEQ ID NO: 147 - protein, 8 aa, Homo sapiens
  CDR3 sequence
GWELGLDD

SEQ ID NO: 148 - protein, 11 aa, Homo sapiens
  CDR3 sequence
GWGSYFHAFDI

SEQ ID NO: 149 - protein, 7 aa, Homo sapiens
  CDR3 sequence
GWYASDI

SEQ ID NO: 150 - protein, 7 aa, Homo sapiens
  CDR3 sequence
GYYDMDV

SEQ ID NO: 151 - protein, 12 aa, Homo sapiens
  CDR3 sequence
HEALMTTWLLDV

SEQ ID NO: 152 - protein, 10 aa, Homo sapiens
  CDR3 sequence
HPGELGAFDI

SEQ ID NO: 153 - protein, 12 aa, Homo sapiens
  CDR3 sequence
HSDARWPPNFDY

SEQ ID NO: 154 - protein, 17 aa, Homo sapiens
  CDR3 sequence
NLGHDFWSGYYHDAFDI

SEQ ID NO: 155 - protein, 13 aa, Homo sapiens
  CDR3 sequence
QEGLVDSYYGMDV

SEQ ID NO: 156 - protein, 13 aa, Homo sapiens
  CDR3 sequence
RFRYSSSSDVFDI

SEQ ID NO: 157 - protein, 13 aa, Homo sapiens
  CDR3 sequence
RFWYSSSSDVFDI

SEQ ID NO: 158 - protein, 16 aa, Homo sapiens
  CDR3 sequence
RGSGHGSYYYFHTMDV

SEQ ID NO: 159 - protein, 15 aa, Homo sapiens
  CDR3 sequence
RHDSGKYRYHDAFDI

SEQ ID NO: 160 - protein, 10 aa, Homo sapiens
  CDR3 sequence
RHESLNAFDV

SEQ ID NO: 161 - protein, 10 aa, Homo sapiens
  CDR3 sequence
RHLLLDVFDV

SEQ ID NO: 162 - protein, 18 aa, Homo sapiens
  CDR3 sequence
RSGYGSGPVYYYHYGMDV

SEQ ID NO: 163 - protein, 18 aa, Homo sapiens
  CDR3 sequence
RSYYSSSLQREIHYGMDV

SEQ ID NO: 164 - protein, 17 aa, Homo sapiens
  CDR3 sequence
SAEHWVAPNYYFHNMDV

SEQ ID NO: 165 - protein, 16 aa, Homo sapiens
  CDR3 sequence
TESSGSSPYYYHYMDV

SEQ ID NO: 166 - protein, 14 aa, Homo sapiens
  CDR3 SEQUENCE
TTGKQQLPRGAFDI

SEQ ID NO: 167 - protein, 10 aa, Homo sapiens
  CDR3 sequence
VDTLTKAFDV

SEQ ID NO: 168 - protein, 13 aa, Homo sapiens
  CDR3 sequence
VFRYSSSSDVFDI

SEQ ID NO: 169 - protein, 11 aa, Homo sapiens
  CDR3 sequence
VRSGPYDPFDI

SEQ ID NO: 170 - protein, 7 aa, Homo sapiens
  CDR3 sequence
WIQPFDY

SEQ ID NO: 171 - protein, 7 aa, Homo sapiens
  CDR3 sequence
WLQPFDY

SEQ ID NO: 172 - protein, 12 aa, Homo sapiens
  CDR3 sequence
YGVVGGRRYFDY

(19) United States
(12) Patent Application Publication
(10) Pub. No.: US 2017/0306039 A1
(43) Pub. Date: Oct. 26, 2017

(54) HUMAN VH DOMAIN SCAFFOLDS

(71) Applicant: Crescendo Biologics Limited, Cambridge, Cambridgeshire (GB)

(72) Inventors: Bryan Edwards, Cambridge, Cambridgeshire (GB); Mingyue He, Cambridge, Cambridgeshire (GB)

(73) Assignee: Crescendo Biologics Limited, Cambridge, Cambridgeshire (GB)

(21) Appl. No.: 15/520,639

(22) PCT Filed: Oct. 22, 2014
(86) PCT No.: PCT/GB2014/053145

§ 371 Date: Apr. 20, 2017

(51) Int. Cl. C07K 16/28; C07K 16/46; C12N 15/10; C07K 16/00; C07K 16/24

(52) U.S. Cl. CPC C07K 16/2878; C07K 16/005; C07K 16/241; C07K 16/46; C12N 15/1041; C07K 16/00; C07K 2317/21; C07K 2317/569; C07K 2317/76; C07K 2317/94; C07K 2318/10; C07K 2317/565

(57) ABSTRACT

The invention provides human VH scaffold sequences, libraries derived therefrom and methods of producing. The scaffolds have high expression, solubility and are functional.

### FIELD OF THE INVENTION

The invention relates to novel VH domain scaffolds, libraries derived from the scaffolds, methods of construction and pharmaceutical compositions comprising the VH domain scaffolds.

### BACKGROUND TO THE INVENTION

Most natural conventional antibodies or immunoglobulins (Ig's) are tetrameric molecules made up of paired heterodimers (each comprising one heavy and one light chain) stabilised and cross-linked by inter-chain and intra-chain disulphide bonds. The light chains may be of either the kappa or lambda isotype. Each of the heavy and light chains fold into domains, each light chain having an N-terminal variable (VL) and a C-terminal constant domain (CL) which may be either Cκ or Cλ. Each heavy chain comprises an N-terminal variable (VH) domain followed by a first constant domain (CH1) a hinge domain and two or three further constant domains (CH2, CH3 and optionally CH4). Association of the VH domain on each heavy chain with the VL domain on its partner light chain results in the formation of two antigen binding regions (Fv). Interaction between the CH1 domain and the CL domain is known to facilitate functional association between the heavy and light chains. Each Fv region comprises an antigen binging site formed by six hypervariable polypeptide loops or complementarity determining regions (CDRs), three derived from the VH domain (H1, H2 and H3) and three from the VL domain (L1, L2 and L3). The CDRs interact directly with antigen. The scaffold sequences in the Fv which support the CDRs are known as framework regions (FRs).

The VH domain is encoded by gene segments located in the heavy chain locus. Similarly the VL domain is encoded by gene segments located in one of the two light chain loci. During normal B-cell development, one of a multitude of VH gene segments is rearranged with one of a number of D-gene segments and one of a number of J-gene segments, the final VDJ arrangement encoding a complete VH region. The majority of the VH region (including CDRs 1 and 2) is encoded by the VH gene segment. The D-J combination encodes the rest of the VH region (in particular CDR3). Combinatorial choice of exactly which V-, D- and J-gene-segments are used, imprecision of the D-J join and somatic hypermutation all result in significant sequence diversity focused in the heavy chain CDRs. In particular, the heavy chain CDR3 acquires greatest sequence diversity and therefore generally contributes the most to antibody specificity. The light chains undergo a similar process, recombining one light chain V-gene segment with one light chain J-gene segment to form the VL sequence. Combinatorial sequence diversity is once again focused in the VL CDRs.

The constant regions of both the heavy and light chains are relatively invariant.

In conventional antibodies, generally, both the VH and the VL are required for antigen binding. However, camelids (camels, dromedaries and llamas) and certain sharks are known to naturally produce a class of functional antibodies devoid of light chains (Hamers et al 1993). Such heavy-chain only antibodies are distinct from conventional antibodies in that they are homodimers of a heavy chain comprised of a VH and a number of CH domains but importantly they lack a CH1 domain. Camelids, are capable of producing both conventional and heavy-chain only antibodies in response to antigen challenge (indeed they often produce both classes of antibody in a single response to antigen). When raising heavy-chain only antibodies, rather than the standard VH domain, camelids use a special class of heavy chain variable region known as VHH (De Genst et al Dev. Comp. Immunol. 30: 187-198).

However, despite many attractive biophysical characteristics, camelid VHH domains do not have a human amino acid sequence and therefore have the potential to initiate an anti-drug immune response when administered to humans. In view of this, VHH domains are not suitable as effective therapeutic products and significant efforts have been made to overcome the problem by ‘humanising’ the camelid sequence. Importantly, it is frequently the case that in order to avoid loss of binding affinity, specificity and functionality it is necessary to retain many original camelid residues. As such, the product destined for therapeutic use in humans will always retain non-human residues.

Consequently there has been a great deal of interest in producing human VH (or VL) domains as therapeutic candidates. It is well known that VH domains derived from conventional antibodies require a companion VL domain and in the absence of the partner domain are difficult to express, often insoluble and suffer loss of binding affinity and specificity to target antigen.

Isolated human VH (or VL) domains require significant engineering in order to enhance solubility and stability. This problem has been approached in a number of ways, for example by ‘camelising’ the human sequence (Davies and Reichmann 1996 Protein Eng 9(6):531-537; Reichmann L and Muyldermans S 1999 J Immunol Methods 231:25-38). Indeed, the requirement for significant engineering to enhance solubility and stability of isolated human VH (or VL) domains means that deriving drug quality therapeutic candidates has been extremely challenging.

Libraries of the prior art have attempted to overcome these limitations, for example US 2011/0052565 describes libraries of non-aggregating human VH domains comprising at least one di-sulphide cysteine in at least one CDR and having an acidic isoelectric point. Non-aggregating VH domains are selected using a heat denaturation and refolding step since selection based solely on binding was not efficient in yielding functional binders. EP1025218 describes a naïve library of human VH domains, all members having a H1 hypervariable loop canonical structure encoded by VH gene segment DP-47, wherein loop is diversified by changing aa at positions H31, H33 and H35. Each time the VH libraries of EP1025218 are used for selecting on target antigen, they are first screened in accordance with the ability to bind to superantigen protein A, a generic ligand which essentially depletes the library of non-functional or poorly folded members. Subsequent to protein A screening, the depleted antibody repertoire is selected against the target antigen, and further rounds of enrichment for binding to target antigen are performed. Despite the use of a known functional VH3 gene (DP-47) as the basis for a library, the requirement to remove non-functional members prior to initial selection on any target antigen suggests that the initial repertoire contained a significant number of defective clones.

Thus, the VH libraries of the prior art are limited by their ability to yield soluble functional clones without additional steps such as protein A selection, the combination of heat denaturation with refolding or significant prior engineering for enhanced solubility and stability. In view of these limitations there is a need to provide further VH domain libraries comprising high numbers of soluble, functional clones which may be selected in a direct and efficient manner.

Conventional antibodies are now well established as highly effective therapeutic agents with sales of $54 bn in 2012 expected to continue to grow significantly in the coming years. However, there is increasing demand for exploiting the benefits of alternative formats and smaller fragments in order to derive the next generation of antibody-based therapeutic candidates and in light of this and in view of the above-mentioned problems, there is a need to provide further human VH scaffolds, human VH libraries based on the scaffolds and methods thereof which enable the isolation of soluble, stable, high affinity antibodies with low immunogenicity. The provision of further scaffolds and libraries thereof increases the diversity of potential antibodies that may be obtained against a particular target antigen and therefore increases the probability of isolating a VH domain with the desired affinity and specificity. The scaffolds of the present invention provide a valuable contribution to the art and further advance the repertoire of soluble human VH domains available to be screened and progressed for clinical development.

### SUMMARY OF THE INVENTION

According to a first aspect of the invention there is provided a human VH scaffold capable of producing a VH domain expression library comprising at least 70% soluble clones. The clones are highly expressed, functional and non-aggregating. The clones may be further characterised by the presence of a single, monomer peak when purified by size exclusion chromatography. The scaffolds provide new soluble frameworks for the generation of a diverse VH domain expression library.

In one embodiment of the invention there is provided human VH scaffolds or fragments thereof according to Seq ID No. 1, Seq ID No. 2, Seq ID No. 3, Seq ID No. 4, Seq ID No. 5 and Seq ID No. 6.

According to a further aspect of the invention there is provided a method for identifying a VH scaffold comprising the steps of:
a) Obtaining a human VH domain expression library
b) Screening the library of step a) against protein A
c) Identifying VH domains which bind protein A and expressing in E. coli
d) Detecting soluble VH domains expressed in step c)
e) Determining the sequence of soluble VH domains to obtain a VH scaffold sequence.

According to a further aspect of the invention there is provided human VH domain expression libraries derived from the scaffolds of the invention. The libraries comprise a population of VH clones having at least 70% solubility, are highly expressed, functional and non-aggregating. The libraries are useful in providing for direct and efficient isolation of VH domain antibodies.

In one embodiment there is provided human VH domain expression libraries derived from the scaffolds according to Seq ID No. 1, Seq ID No. 2, Seq ID No. 3, Seq ID No. 4, Seq ID No. 5 and Seq ID No. 6.

According to a further aspect of the invention there is provided a method of constructing a VH domain expression library comprising the steps of;
a) Assembling the scaffolds according to the first aspect to comprise CDR3 regions
b) Obtaining a VH domain repertoire
c) Expressing the VH domain repertoire and selecting for functional VH domains against target antigen.

In one embodiment there is provided a method of constructing a VH domain expression library comprising the steps of;
a) Assembling the scaffolds as defined according to the previous aspects to comprise CDR3 regions
b) Obtaining a VH domain repertoire
c) Expressing the VH domain repertoire and selecting for functional VH domains against target antigen.

In a further aspect of the invention there is provided an isolated human VH domain or fragment thereof comprising a scaffold as defined in the previous aspects. The invention further relates to a VH domain or fragment thereof derived comprising a scaffold as defined in the previous aspects wherein the VH domain does not bind protein A.

In a further aspect of the invention there is provided a pharmaceutical composition comprising a therapeutically effective amount of a VH domain antibody derived from the VH libraries of the invention, and a pharmaceutically acceptable excipient.

In a further aspect of the invention there is provided a method of treatment by administering an effective amount of the VH domain of the present invention to an animal.

### BRIEF DESCRIPTION OF THE FIGURES

FIG. 1 shows PCR amplification of (a) human VH domains from cDNA (lanes 1-3) and (b) human Cκ fragment. (lanes 4 to 7). PCR amplification products were observed at the expected size (approx 300-400 bp for both products, arrowed)

FIG. 2 shows PCR amplification of full-length human VH domains assembled with human Cκ fragment. PCR amplification products were observed at the expected size (approx 700 bp, arrowed)

FIG. 3 shows recovery of protein A binding VH fragments from ribosome display selections. PCR amplification products were observed at the expected size (approx 700 bp, arrowed) on the protein A selections but not on selections with BSA.

FIG. 4 shows PCR amplification of full-length human VH fragments assembled with N-terminal T7 promoter and C-terminal 6× histidine tag. PCR amplification products were observed at the expected size (approx 500 bp, arrowed)

FIG. 5 shows a 96 well dot blot of human VH expressed in E. coli. Soluble VH were detected using anti-HIS HRP. VH-H-3 and V3-93 VH are arrowed.

FIG. 6 shows SDS-PAGE of E. coli extracts expressing VH-H-3 and VH3-93 VH. Left=total extracts, right=VH fragments following affinity purification by nickel agarose chromatography.

FIG. 7 shows PCR amplification of human CDR3 domains from cDNA. CDR3 amplification products were observed at the expected size (approx 50 to 100 bp, arrowed)

FIG. 8 shows assembly and pull-through PCR amplification of V3-93 scaffold plus human CDR3 domains. Full length VH products were observed at the expected size (approx 400 bp, arrowed)

FIG. 9 shows a schematic diagram of phagemid vector pUCG3

FIG. 10 shows PCR amplification of pUCG3 vector. A PCR product was observed at the expected size (approx 4600 bp, arrowed).

FIG. 11 shows solubility of VH clones from the VH-H-3 library (left) and V3-93 library (right).

FIGS. 12a and 12b show clones from the V3-93 and VH-H-3 libraries respectively having solubility of at least 70%.

FIG. 13 shows VH yields following purification from small scale expression studies by affinity chromatography across all antigens.

FIG. 14 shows calibration of HPLC with known standards

FIG. 15 shows SEC profile of anti-TNFR1 VH isolated from V3-93 library (46H6, left) and VH-H-3 library (56B7, right)

FIG. 16 shows anti-TNFR1 VH (38H9, 44B8, 46E12, 46H6) inhibit binding of TNF-α to TNFR1 in a competition binding assay. C170=anti-TNF-α reference dAb.

FIG. 17 shows VH 81G1, 46H6, 74B10, 82B4 and 46G8 binding to antigens hTNFR1, hTRAIL, hFas, hNGFR, hTNFR2, shTNFR1, KLH and ovalbumin in phage ELISA.

FIG. 18 shows amino acid alignment of anti-TNFR1 VH sequences with human VH3-23 (DP47). When this panel of VH were tested by ELISA (FIG. 19), 81G1 was the only VH not to bind to protein A because of a mutation in the protein A binding site (Kabat H82b Asn to Asp, arrowed).

### DETAILED DESCRIPTION OF THE INVENTION

The inventors have provided new VH scaffolds that form the basis for the construction of diverse libraries of VH domains which retain the advantageous features of the scaffolds, and are soluble, non-aggregating, correctly folded, stable and functional.

### Scaffolds

According to a first aspect of the invention there is provided a human VH scaffold or fragment thereof capable of producing a VH domain expression library comprising at least 70% soluble clones. The presence of soluble clones may be measured by analysis of bacterial periplasmic extracts using techniques known in the art, for example immunoblotting or ELISA. With the appropriate leader sequences present, soluble VH expressed in E. coli are transported to the bacterial periplasmic space. Here they can be extracted and coated directly onto solid supports for detection by ELISA. When using ELISA, the absorbance at 450 nm is directly proportional to the amount of VH coated, and therefore gives an indication of VH expression and solubility. The inventors have found that the proportion of clones derived from the libraries of the invention which are defined as soluble according to a reading of between 0.2 and 3 OD at 450 nm in ELISA is at least 70%.

Solubility is known to the skilled person as the maximum amount of solute dissolved in a solvent at equilibrium and may also be referred to herein as the ability of a VH domain to dissolve in an appropriate buffer such as phosphate buffered saline (PBS), Tris buffers, HEPES buffers, carbonate buffers or water and to bind antigen.

VH domains are monomeric and in the absence of a VL partner are characteristically “sticky” tending to form aggregates in solution and binding non-specifically to antigen caused by the exposure of hydrophobic amino acid residues that would normally interact with the light chain. This problem is recognised in the prior art and can result in a decrease in the quality and diversity of a library. The VHs of the invention are monomeric in form and do not form aggregates in solution. This is due to the properties of the scaffold sequence which in effect act as a template, transferring their inherent properties such as high solubility, low propensity to aggregate, stability and functionality to the VH domain antibodies produced from them. The presence of a stable, soluble VH domain in monomeric form may be confirmed by the presence of a single correct peak following size exclusion chromatography (SEC).

The scaffolds of the invention have been found to result in the isolation of a higher proportion of soluble and correctly folded VH domains from a VH library based on the scaffolds as defined herein. The scaffolds of the invention are capable of producing a VH domain expression library comprising at least 70% soluble clones which are non-aggregating as defined according to the presence of a single correct monomer peak following size exclusion chromatography (SEC), and are stable and functional as defined by the ability to bind antigen.

The scaffolds of the invention provide new soluble frameworks for the generation of diverse VH domain libraries which do not require additional modifications such as protein A depletion prior to selection on each target antigen in order to reduce background levels due to significant numbers of non-functional clones.

The term “VH” or “VH domain” as used herein refers to an antibody heavy chain variable domain. This includes human VH domains and VH domains that have been altered, for example by mutagenesis and those which occur naturally.

In one embodiment of the invention there is provided human VH scaffolds or fragments thereof according to Seq ID No. 1, Seq ID No. 2, Seq ID No. 3, Seq ID No. 4 Seq ID No. 5 and Seq ID No. 6.

In another embodiment the invention provides a human scaffold or fragment thereof according to Seq ID No. 1 and Seq ID No. 4. The scaffold is derived from the human VH germline sequence V3-23 (Identified in VBASE2 at http://www.vbase2.org/vgene.php?id=humIGHV187; Retter I et al Nucl. Acids Res. (2005) 33 (suppl 1): D671-D674) and is referred to herein as VH-H-3.

In another embodiment the invention provides a human scaffold or fragment thereof according to Seq ID No. 2 and Seq ID No. 5. The scaffold is derived from the human VH germline sequence V3-23 (Identified in VBASE2 at http://www.vbase2.org/vgene.php?id=humIGHV187; Retter I et al Nucl. Acids Res. (2005) 33 (suppl 1): D671-D674) and is referred to herein as V3-93.

In another embodiment the invention provides a human scaffold or fragment thereof derived from clone 81G1 according to Seq ID No. 3 and Seq ID No. 6. The scaffold is derived from human VH germline sequence V3-23 (Identified in VBASE2 at http://www.vbase2.org/vgene.php?id=humIGHV187; Retter I et al Nucl. Acids Res. (2005) 33 (suppl 1): D671-D674). Following shuffling of both CDR1 and CDR2 of V3-93 and selection on target antigen, the inventors have identified a new VH antibody which differs from the parent V3-93 by a single mutation at Kabat position H82b, Asn to Asp referred to herein as clone 81G1. Scaffold 81G1 is derived from the VH antibody referred to as clone 81G1 which in turn is derived from clone 46H6 as described in the examples herein. The Kabat numbering system is well known to the person skilled in the art and refers to the system used for numbering residues in immunoglobulins and providing a standardised way of identifying residues corresponding to individual domains such as the heavy or light chain variable domains from the compilation of antibodies according to Kabat et al., Public Health Service, National Institutes of Health, Bethesda, Md. (1991).

Scaffolds VH-H-3 and V3-93 were isolated from a VH domain library made by the inventors derived from human spleen cDNA and expressed and screened using ribosome display technology (EP0985032; Hanes, J., Pluckthun, A., Proc. Natl. Acad. Sci. USA; 1997; 94(10): 4937-4942; Irving, R A et al, J Immunol. Methods; 2001; 1; 248(1-2): 31-45). The library was screened against protein A and two scaffolds defined as VH-H-3 and V3-93 were identified as being soluble. The techniques used are known to the skilled person in the art and the method is summarised in the examples described herein.

Protein A is described as a generic ligand in that any antibody which is properly folded and expressed will bind to it. Protein A binding has been used to determine VH domains that retain the necessary characteristics of folding, expression and functionality and allows for a VH library to be depleted of those VH domains that do not have these properties thereby enriching for properly folded and expressed VH domains prior to initial selection on any target antigen (EP1025218).

Protein A is found in the cell wall of the bacterium Staphylococcus aureus and has the ability to bind immunoglobulins, particularly IgGs. It binds to the Fc region of immunoglobulin heavy chains but also to the Fab region in the case of the human VH3 family. Protein A has found a number of uses in scientific research, particularly as a tool for the purification of IgG molecules or fusion proteins expressed with an Fc domain. However, Fc-fusion proteins purified by protein A affinity chromatography often carry residual amounts of protein A that has leeched off of the affinity column during purification. This residual protein A can often cause problems in downstream processes, for example when performing selections with phage display libraries containing human VH3 fragments as these will bind to protein A irrespective of their antigen binding specificity.

Issues caused by the presence of Protein A can be resolved by either, (1) depleting the residual Protein A using methods such as IgG affinity chromatography or, (2) by developing a variant of the human VH3 family that lacks Protein A binding capability.

Despite the high sequence identity to DP-47 the inventors have surprisingly found that VH antibody 81G1 does not bind to protein A, but still retains good solubility and expression characteristics. Often antigen preparations are contaminated with protein A and can cause non-specific binding. The inventors noted (as shown in FIG. 19) that VH antibody 81G1 does not possess the characteristic non-specific binding associated with VH3-derived antibodies. VH antibody 81G1 is derived from anti-TNFR1 VH antibody 46H6 which has undergone CDR1 and CDR2 mutagenesis. Scaffold 81G1 is derived from VH antibody 81G1. The inventors have provided a new scaffold that may be used to derive libraries that do not need to be screened against protein A in order to facilitate the identification of functional antibodies, thereby maximizing library quality and diversity and avoiding the problems associated with protein A antigen contamination. Hence, there is provided a human VH scaffold having the advantage that libraries comprising VH domains based on this scaffold comprise a high proportion of functional, correctly folded members and provide VH domains that may be screened accurately and reliably against target antigens without the need for a protein A enrichment step prior to selection on each target antigen.

The scaffolds of the invention are suitable for the generation of a diverse VH domain library.

All the scaffolds described are derived from the human germline gene V3-23.

The scaffolds as defined herein may be referred to as comprising CDR regions 1 and 2, (CDR1 and CDR2). The scaffolds may be further modified to comprise CDR3 regions, thus forming a diverse library of VH domains comprising CDR1, CDR2, CDR3 and framework regions (FR1, FR2, FR3 and FR4). The framework regions are known as those regions that represent the structural element of the FV region, outside of the CDR regions.

The framework regions of the scaffold may comprise one or more mutations. The mutations may be in any region of the framework region sequence.

The CDR1 and CDR2 regions of the scaffold may be mutated to improve the characteristics of the VH domain, for example improved affinity, solubility, expression or reduced aggregation. Further diversity may be introduced by general molecular biology techniques known to those skilled in the art including site directed mutagenesis, random mutagenesis, error-prone PCR, insertions and deletions (Ausubel et al, Current Protocols in Molecular Biology, John Wiley & Sons, New York 2000).

The invention comprises VH scaffold sequences having at least 80%, 90%, 95%, 98% or 99% amino acid sequence identity with the sequences according to Seq ID No. 1, Seq ID No. 2 or Seq ID No. 3. Percent (%) sequence identity can be determined by methods known in the art. For example mathematical algorithms may be employed to compare amino acid sequence similarity between aligned sequences (Karlin & Altschul, Proc. Natl. Acad. Sci. USA 1990; 87: 2264-2268). Various other programs and software packages may be used including the ALIGN program and the FASTA algorithm (Pearson & Lipman, Proc. Natl. Acad. Sci. USA 1988; 85: 2444-2448). The BLAST program provided by the National Center for Biotechnology Information is also widely used and suitable for the purposes of the present invention.

The scaffolds of the invention comprise CDR1 and CDR2 sequences having at least 80%, 90%, 95%, 98% or 99% amino acid sequence identity with the CDR1 and CDR2 sequences according to Seq ID No. 1, Seq ID No. 2 or Seq ID No. 3. Alternatively the scaffolds of the invention comprise one of CDR1 or CDR2 sequences having at least 80%, 90%, 95%, 98% or 99% amino acid sequence identity with the CDR1 and CDR2 sequences according to Seq ID No. 1, Seq ID No. 2 or Seq ID No. 3.

The invention also relates to nucleic acid sequences encoding the VH scaffold scaffolds having at least 80%, 90%, 95%, 98% or 99% sequence identity with the sequences according to Seq ID No. 4, Seq ID No. 5 or Seq ID No. 6.

The invention further relates to CDR1 and CDR2 nucleic acid sequences having at least 80%, 90%, 95%, 98% or 99% sequence identity with the CDR1 and CDR2 sequences according to Seq ID No. 4, Seq ID No. 5 or Seq ID No. 6. Alternatively the scaffolds of the invention comprise one of CDR1 or CDR2 nucleic acid sequences having at least 80%, 90%, 95%, 98% or 99% sequence identity with the CDR1 and CDR2 sequences according to Seq ID No. 4, Seq ID No. 5 or Seq ID No. 6.

The scaffolds of the invention may comprise one or more CDR1 and CDR2 sequences which are grafted in to replace one or both of the existing CDR regions and may be derived from non-human sources, for example camel or mouse. For example the VH domain may comprise a human framework region and a camelid CDR1 and/or CDR2 region. Alternatively the scaffolds may comprise humanised CDR1 and/or CDR2 sequences derived from non-human species such as camel or mouse.

According to a further embodiment there is provided a method for identifying a VH scaffold of the first embodiment comprising the steps of:
a) Obtaining a human VH domain expression library
b) Screening the library of step a) against a generic ligand
c) Identifying VH domains which bind the generic ligand and expressing in E. coli
d) Detecting soluble VH domains expressed in step c)
e) Determining the sequence of soluble VH domains to obtain a VH scaffold sequence.

In one aspect, there is provided a method for identifying a VH scaffold comprising the steps of:
a) Obtaining a human VH domain expression library
b) Screening the library of step a) against a generic ligand
c) Identifying VH domains which bind the generic ligand and expressing in E. coli
d) Detecting soluble VH domains expressed in step c)
e) Determining the sequence of soluble VH domains to obtain a VH scaffold sequence.

The method comprises the step of screening the VH domain expression library of step a) against a generic ligand to identify a soluble VH domain scaffold that may form the basis for a VH domain expression library. The step of screening against a generic ligand in this manner enables libraries to be constructed which retain the soluble characteristics of the scaffold. The inventors have found that expression libraries derived from the scaffold which was identified using this method, comprise a population of VH clones having at least 70% solubility. The high proportion of soluble clones in the library means that it is not a requirement to deselect the VH expression library against a generic ligand prior to screening against target antigen. The method therefore provides for soluble VH domains to be isolated from a VH expression library in an efficient and high throughput manner against target antigen without the need for pre-screening against a generic ligand.

The VH domain library may be obtained from sources known to the person skilled in the art for example spleen or bone marrow. The VH library may be expressed by any conventional techniques known in the art, for example phage display, ribosome display technology, yeast display, microbial cell display or expression on beads such as microbeads. In one aspect the VH domains are expressed using ribosome display technology (EP0985032; Hanes, J., Pluckthun, A., Proc. Natl. Acad. Sci. USA; 1997; 94(10); 4937-4942; Irving, R A et al, J. Immunol. Methods; 2001; 1; 2489(1-2); 31-45).

The library may be screened against any known generic ligand which binds to an expressed VH polypeptide irrespective of the specificity of the VH polypeptide for antigen. In one aspect the generic ligand is protein A.

Soluble, expressed VH domains may be detected using techniques known in the art, for example immunoblotting, ELISA or by direct purification by affinity chromatography. In one aspect the VH domains are detected by immunoblotting.

The sequences of identified soluble VH polypeptides are determined using methods known in the art. The VH domain polypeptide identified in step e) comprises a CDR3 region, therefore to determine the sequence of the scaffold, the CDR3 sequence is removed.

### Libraries

According to a further aspect of the invention there is provided human VH domain expression libraries derived from the scaffolds of the invention. The libraries comprise a population of VH clones having at least 70% solubility, are highly expressed, functional and non-aggregating. The libraries have the advantage of providing for direct and efficient isolation of VH domain antibodies.

In one embodiment there is provided human VH domain expression libraries derived from the scaffolds according to Seq ID No. 1, Seq ID No. 2, Seq ID No. 3, Seq ID No. 4, Seq ID No. 5 and Seq ID No. 6.

According to a further embodiment of the invention there is provided a method of constructing a VH domain expression library comprising the steps of;
a) Assembling the scaffolds according to the first aspect with a plurality of CDR3 nucleic acid sequences to obtain a VH domain repertoire
b) Expressing the VH domain repertoire to produce a VH domain library and selecting for functional VH domains against target antigen.

In a further embodiment there is provided a method of constructing a VH domain expression library comprising the steps of;
a) Assembling the scaffolds according to Seq ID No. 1, Seq ID No. 2, Seq ID No. 3, Seq ID No. 4, Seq ID No. 5 or Seq ID No. 6. of CDR3 nucleic acid sequences to obtain a VH domain repertoire,
b) Expressing the VH domain repertoire to produce a VH domain library and selecting for functional VH domains against target antigen.

The method may comprise an additional modification step, for example CDR3 mutagenesis followed by further rounds of screening against target antigen. This may improve VH domain characteristics such as solubility and immunogenicity.

The method may comprise the additional step of sequencing the selected VH domains.

The method may further comprise the additional step of expressing the selected VH domain in a host cell. Typical examples of host cells include E. coli in particular TG1, BL21(DE3), W3110 and BL21(DE3)pLysS.

The VH domain repertoire may be expressed by any known method in the art, for example phage display or ribosome display as described herein.

The libraries comprise the VH domain scaffolds and enable VH domains which have the advantageous properties of the scaffold including solubility, stability and functionality to be obtained.

In one aspect the invention provides a VH domain library comprising the scaffold sequence according to Seq ID No. 1 and is referred to herein as VH-H-3.

In another aspect the invention provides a VH domain library comprising the scaffold sequence according to Seq ID No. 2 and referred to herein as V3-93.

In a further aspect the invention provides a VH domain library comprising the scaffold sequence according to Seq ID No. 3 and referred to herein as scaffold 81G1.

CDR3 regions are known to have the most variability in comparison with CDR1 and CDR2 domains and therefore enable the generation of a library containing at least 10⁹ or more unique VH domains with a common structural framework or scaffold. In a further embodiment the invention comprises libraries comprising at least 10⁹, 10¹⁰, 10¹¹ or 10¹² unique VH domains.

The CDR3 region to be introduced may be derived from any source including human, non-human, synthetic and humanised. CDR3 regions are known to vary in size and typically are between 4 to 25 amino acid residues in length. Typically a CDR3 region is approximately 12 amino acids in length. A humanised antibody repertoire comprises antibodies which are derived from a non-human source and have been modified by the mutation of certain amino acid residues to make the antibody more human-like, for example to impart low immunogenicity characteristics. The number of amino acid residues mutated may vary depending on the desired characteristics. In one embodiment the CDR3 region is derived from a naïve or non-immunized source and may be human, humanised or non-human. A naïve repertoire or library is derived from a source where the animal has not been exposed to antigen. In one example the CDR3 region is derived from a camelid or mouse naïve repertoire. In one example the CDR3 region is human and derived from a naïve repertoire for example peripheral blood lymphocytes, spleen, lymph node, peripheral blood or bone marrow. In a further example the CDR3 region is synthetic or humanised.

The CDR3 region to be introduced may be derived from an immunised source. An immunised repertoire derived from a human or non-human animal which has been exposed to antigen and as a result the repertoire contains antibodies that recognise the antigen. In one example the CDR3 region is derived from a camelid or mouse immunised repertoire. In a further example the CDR3 region is derived from a human immunised repertoire, for example from peripheral blood lymphocytes, spleen, lymph node or bone marrow.

The CDR3 regions may be obtained from commercially available cDNA libraries.

The CDR3 regions may be introduced into VH scaffold by any suitable method known in the art for example PCR (polymerase chain reaction)-based assembly and amplification using primers overlapping the framework and CDR3 regions. VH scaffold containing CDR3 regions may be introduced into any suitable vector (for example a phagemid vector) by any suitable method known in the art for example by PCR-based assembly using a mixture of appropriately linearized vector plus DNA encoding VH scaffold containing CDR3 insert followed by PCR amplification using primers overlapping the framework and CDR3 regions. Evaluation of the VH clones is performed for example by ELISA (Enzyme Linked Immunosorption Assay) following expression using a suitable vector in a host cell, for example E. coli.

The CDR3 regions may be subject to further mutagenesis after introduction into the scaffolds of the invention. This offers the advantage that the library may be tailored or biased towards a target antigen after an initial round of selection against that antigen to obtain VH domains offering improved affinity, solubility or expression. Alternatively the CDR3 regions may be subject to one or more rounds of mutagenesis prior to selection against antigen. In addition to tailoring the VH library to a particular antigen, further mutagenesis serves to increase the overall size of the repertoire thereby increasing the likelihood of obtaining an antibody with the desired characteristics.

The mutagenesis methods used to introduce further diversity represent general molecular biology techniques known to those skilled in the art including site directed mutagenesis, random mutagenesis, error-prone PCR, insertions and deletions (Ausubel et al, Current Protocols in Molecular Biology, John Wiley & Sons, New York 2000).

CDR1, CDR2 and/or CDR3 regions of the VH domains of the invention may comprise one or more acidic amino acids to improve solubility and/or reduce aggregation. Typically the VH domains may comprise Asp or Glu at position 32 of CDR1.

Once the library has been assembled following the introduction of CDR3 regions in a suitable expression vector, the VH domains are expressed for screening against a target antigen. The library may be expressed and screened by any conventional techniques known in the art for example phage display, ribosome display, yeast display, microbial cell display or expression on beads such as microbeads. In one embodiment the library is expressed by any selection display system which permits the nucleic acid of a VH domain to be linked to the expressed VH polypeptide, for example phage display systems wherein VH domains are expressed on the surface of filamentous bacteriophage and screened against target antigen (McCafferty, J., Griffiths, A D., Winter, G., Chiswell, D J, Nature, 348 1990; 552-554). The bacteriophage library may be screened against antigen using techniques well known in the art (for example as described in Antibody Engineering, Edited by Benny Lo, chapter 8, p 161-176, 2004) which may be immobilised (for example attached to magnetic beads or on the surface of a microtitre plate) or expressed on the surface of a cell, in solution or in any other format. The skilled person will be aware that the target antigen may be any antigen of interest, for example purified, expressed on the surface of a cell, partially purified or peptides. Typically the target antigen is a purified protein. The library may also be screened against antigen in a high-throughput manner, for example in microarrays. Binding phage are retained, eluted and amplified by infection of E. coli or other suitable host cells and phage isolated and screened again against target antigen. This process can be repeated numerous times, for example 2 to 10 repeats resulting in the enrichment of VH domains specific for the target antigen or until VH domains possessing the desired characteristics are obtained. The gene sequence encoding the VH domain may then be determined using standard techniques for example amplifying the VH nucleic acid sequence and determining the amino acid sequence, cloning the sequence into an expression vector and expressing in E. Co/i, or other suitable host cells to further determine the properties of the isolated VH domain.

Alternatively the VH domain library may be expressed by ribosome display technology wherein the VH are displayed as polypeptides on the surface of a ribosome together with the corresponding mRNA. The ribosome display library may be screened against immobilised antigen (for example attached to magnetic beads or on the surface of a microtitre plate, or using affinity chromatography column with a resin bed containing the ligand). Reverse transcription of mRNA derived from the ribosome/mRNA/polypeptide complex generates the cDNA from which the library is derived. The isolated sequence may then undergo mutagenesis or further rounds of screening in the ribosome display system. The techniques for construction of ribosome display libraries and methods of isolation of antigen binders is well known in the art (EP0985032; Hanes, J., Pluckthun, A., Proc. Natl. Acad. Sci. USA; 1997; 94(10): 4937-4942; Irving, R A et al, J Immunol. Methods; 2001; 1; 248(1-2): 31-45).

The invention further provides isolated human VH domains or fragments thereof comprising a scaffold as defined in the previous aspects. The invention further relates to VH domains comprising a scaffold as defined in the previous aspects. wherein the VH domains do not bind protein A.

Such VH domain antibodies are soluble, non-aggregating, stable and functional. They exhibit high affinity binding to a target antigen.

In one embodiment the VH domain antibodies or fragments thereof are characterised in that they comprise the scaffold sequences as defined herein in accordance with Seq ID No. 1, Seq ID no. 2, Seq ID No. 3. Seq ID No. 4, Seq ID No. 5 or Seq ID No. 6.

The invention encompasses nucleic acids encoding the VH domain antibodies of the invention. The nucleic acid may be double stranded, single stranded, including cDNA or RNA.

The invention also relates to vectors and host cells comprising the nucleic acid sequences encoding the VH domain of the invention. Suitable vectors are known to those skilled in the art. and include pGEX, pDEST, pET, pRSET, pBAD and pQE. Suitable host cells may be eukaryotic or prokaryotic. Preferably the host cells are bacterial for example E. coli. Strains of E. coli known to the skilled person include TG1, BL21(DE3), W3110 and BL21(DE3)pLysS.

The proportion of VH domains in the libraries of the present invention with improved solubility characteristics may be higher compared to similar libraries of the prior art derived from scaffolds with lower solubility characteristics. The inventors have determined that the proportion of soluble clones present in the libraries described herein is at least 70%.

The VH domains or fragments thereof may be isolated and purified from the host cells expressing them by techniques known in the art. Purification of VH domains as referred to herein may be carried out by suitable methods known in the art. For example the VH domains may be purified from the host cell or cell culture medium by chromatography, ion-exchange chromatography, size exclusion chromatography, high performance liquid chromatography (HPLC) and affinity chromatography (Methods in Enzymology, Vol. 182, Guide to Protein Purification, Eds. J. Abelson, M. Simon, Academic Press, 1st edition, 1990).

Further to purification the VH domain may undergo genetic modifications such as mutagenesis in one or more of the CDR regions using standard techniques to improve affinity, solubility or expression, for example site-directed mutagenesis, random mutagenesis, insertions or deletions. If the library is derived from a non-human source then the VH domain may require “humanising” to reduce potential immunogenicity reactions when administered in human therapy. In this respect defined amino acid residues are mutated to engineer the VH domain so that it retains binding affinity and conservative non-human residues are substituted.

The VH domains may form multimers comprising two or more VH domains which is known to improve the strength of binding to antigen by virtue of the increased number of antigen binding sites. For example the VH domains may form homodimers, heterodimers, heteromultimers or homomultimers.

The VH domains may be joined to a moiety designed to optimise the PK/PD characteristics of the VH in systemic circulation. In one example the VH domain may be fused directly to the additional moiety and in another example the VH domain may be coupled chemically to the additional moiety either directly or via a linker. The linker may comprise a peptide, an oligopeptide, or polypeptide, any of which may comprise natural or unnatural amino acids. In another example, the linker may comprise a synthetic linker. In one example the additional moiety may be a naturally occurring component (for example serum albumin) or in another example the additional moiety may be polyethylene glycol.

The VH domains may be joined to a toxic moiety with the aim of utilising the binding of the VH domain to its target antigen in vivo to deliver the toxic moiety to an extracellular or intracellular location. The toxic moiety may be fused directly to the VH domain and in another example the toxic moiety may be coupled chemically to the VH domain either directly or via a linker. The linker may comprise a peptide, an oligopeptide, or polypeptide, any of which may comprise natural or unnatural amino acids. In another example, the linker may comprise a synthetic linker.

Further to isolation of the VH domain in accordance with known techniques and as described above, the VH domain may be assayed to determine affinity for the target antigen. This may be carried out by a number of techniques known in the art for example enzyme-linked immunospecific assay (ELISA) and BIAcore (measurement in real time of interactions between molecules using surface plasmon resonance). In addition, binding to cell surface antigens can be measured by fluorescence activated cell sorting (FACS). The affinity of the isolated VH domain indicates the strength of binding to the target antigen and is a crucial parameter in determining whether a candidate VH domain is likely to proceed further into development as a therapeutic. Affinity is commonly measured by the dissociation constant K_{d} (K_{d}=[antibody][antigen]/[antibody/antigen complex]) in molar (M) units. A high K_{d} value represents an antibody which has a relatively low affinity for a target antigen. Conversely a low K_{d}, often in the sub-nanomolar (nM) range indicates a high affinity antibody.

In a further aspect the invention provides a pharmaceutical composition comprising a therapeutically effective amount of a VH antibody derived from the VH libraries of the invention, and a pharmaceutically acceptable excipient. VH antibodies derived from the libraries of the invention possess the desirable characteristics of high solubility, low propensity to aggregate, stability and functionality. Such characteristics allow the VH antibodies to be progressed for therapeutic development and use as diagnostics without the requirement for substantial engineering or modification.

The invention provides pharmaceutical compositions comprising a VH domain in an effective amount for binding to a target antigen and a pharmaceutically acceptable excipient. Suitable pharmaceutically acceptable excipients are known to those skilled in the art and generally includes an acceptable composition, material, carrier, diluent or vehicle suitable for administering the VH domains of the invention to an animal. In this respect the VH domain may be comprised in a whole antibody or fragment thereof. For example the VH domain may be grafted onto a human antibody framework, for example an IgG using methods known in the art.

In a further embodiment the invention provides a method of treatment by administering an effective amount of the VH domain of the present invention to an animal. In this respect the VH domain may be comprised in a whole antibody or fragment thereof. For example the VH domain may be grafted onto a human antibody framework, for example an IgG using methods known in the art.

The invention is described further in the following non-limiting examples.

### EXAMPLES

### Example 1: Identification of Soluble VH-H-3 and VH3-93 Scaffolds by Ribosome Display

### Preparation of Amplified VH Domains

Both VH-H-3 and VH3-93 scaffolds were discovered by ribosome display selections of human VH domains on Protein A. VH domains were amplified from human splenic mRNA by RT-PCR and then assembled with a human Cκ domain as the 3′ end spacer. The stop codon from the human Cκ domain was removed to ensure stalling of the ribosome at the end of translation.

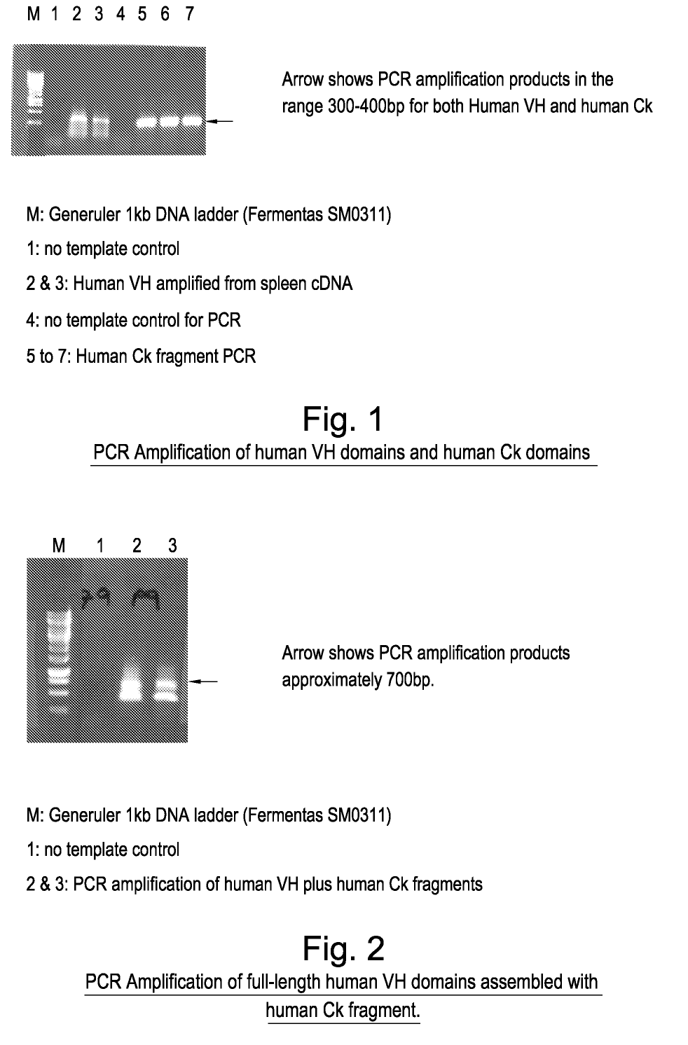

Two primers were designed, T7Ab and VH-ck/F (Table 1), to generate human VH genes flanked by a 5′ T7 promoter plus translation initiation (Kozak) sequence and also a 3′ linker sequence to facilitate joining to human OK. To generate cDNA using the Titan™ system (Boehringer Mannheim), two working solutions were prepared: solution 1 containing 5 μl DTT (100 mM), 2 μl dNTPs (10 mM), 3 μl T7Ab (16 μM), 3 μl VH-cK/F (16 μM) and dH₂O to 50 μl. Solution 2 containing 20 μl 5×RT-PCR buffer (from Titan™ kit) with 28 μl dH₂O. 25 ul of solution 1 was mixed with 25 μl of solution 2 together with 50 ng of splenic mRNA (Invitrogen) and then 0.5 μl of enzyme mix from the Titan™ kit was added. Thermal cycling was carried out using the following programme: 1 cycle of 48° C. for 45 min, followed by 94° C. for 2 min. Then 30-40 cycles of: 94° C. for 30 sec, 54° C. for 1 min, 68° C. for 2 min. Finally, 1 cycle of 68° C. for 7 min for extension, then hold at 10° C. The products of PCR were analysed by agarose gel electrophoresis (FIG. 1) and products around 400 bp purified from the gel using Qiagen gel extraction kit (28704).

TABLE 1
Oligonucleotide primers (5′ to 3′)

| | | Seq ID |
|---|---|---|
| Primer | Sequence | No. |
| T7Ab | GCAGCTAATACGACTCACTATAGGG | 7 |
| | AACAGACCACCATGSARGTNSARCT | |
| | BGWRSAGTCYGG | |
| VH-Ck/F | GCTACCGCCACCCTCGAGTGAAGAG | 8 |
| | ACGGTGACCAGTGTCCC | |
| Link-Ck/B | CTCGAGGGTGGCGGTAGCACTGTGG | 9 |
| | CTGCACCATCTGTC | |
| Ck/F | GCACTCTCCCCTGTTGAAGCT | 10 |
| Ck-f/F | GCACTCTCCCCTGTTGAAGCTCTTT | 11 |
| | GTGACGGGCGAGCTCAGGCCCTGAT | |
| | GGGTGACTTCGCAGGCGTAGAC | |
| T7A1/B | GCAGCTAATACGACTCACTATAGGA | 12 |
| | ACAGACCACCATG | |
| RTKz1 | GAACAGACCACCATGACTTCGCAGG | 13 |
| | CGTAGAC | |
| Kz1 | GAACAGACCACCATG | 14 |
| RTST7/B | GATCTCGATCCCGCG | 15 |
| RTST7/F | CATGGTATATCTCCTTCTTAAAG | 16 |
| Link-His/B | CTCGAGGGTGGCGGTAGCCACCACC | 17 |
| | ACCACCACCAC | |
| Tterm/F | TCCGGATATAGTTCCTCC | 18 |
| RTSN-VH/B | CTTTAAGAAGGAGATATACCATGSA | 19 |
| | RGTNSARCTBGWRSAGTCYGG | |
| T7AB/VH3 | GCAGCTAATACGACTCACTATAGGA | 20 |
| | ACAGACCACCATGGACGAGGTGCAG | |
| | CTGGAGCAGTCTGG | |
| VH3-93/B | GGAACAGACCACCATGGCCCAGGTG | 21 |
| | CAGCTCCAGGAGTCTGG | |
| VHCDR3/B | GGACACGGCCGTGTATTACTGTGC | 22 |
| VHJ/F | GCTACCGCCACCCTCGAGTGARGAG | 23 |
| | ACRGTGACC | |
| pHENAPmut | GTCCATGGCCATCGCCGGCTGGGCC | 24 |
| 4 | GCGAG | |
| pHENAPmut | TAGCAGCCTCGAGGGTGGCGGTAGC | 25 |
| 5 | CATCACCACCATCACCACGGGAGC | |

Preparation of Cκ domain

The human Cκ domain from human IgG was prepared by PCR using primers link-Cκ/B and Cκ/F (Table 1) and a plasmid encoding the Cκ light chain of human IgG (He M. et al. Methods Mol Biol. 2004; 248:177-89). Using Taq DNA polymerase kit from QIAgen (201203), 5 μl 10× buffer, 10 μl 5×Q buffer, 4 μl dNTPs (2.5 mM), 1.5 μl link-Cκ/B (16 μM), 1.5 μl Cκ/F (16 μM), 10 ng of plasmid encoding Cκ, was mixed with dH₂O to 49.75 μl, and 0.25 ul Taq polymerase. Thermal cycling was carried out using the following programme: 30 cycles of 94° C. for 30 sec, 54° C. for 30 sec, 72° C. for 1 min. Finally, one cycle of 72° C. for 7 min for extension, then hold at 10° C. The products of PCR were analysed by agarose gel electrophoresis (FIG. 1) and products around 400 bp purified from the gel using the QIAgen gel extraction kit.

PCR amplification products were observed at the expected size (300-400 bp) for both human VH and human Cκ.

### Assembly of Human VH Domains and Human Cκ Domain

To prepare human VH fragments for ribosome display, the amplified human VH domains were then assembled with DNA encoding the Cκ domain. Equal amounts of the amplified human VH domains were assembled with the Cκ DNA domains by mixing: 2.5 μl 10× buffer, 5 μl 5×Q buffer, 1 μl dNTPs (2.5 mM), 10-50 ng of gel purified human VH domains, 10-50 ng of gel purified human Cκ domain, dH₂O to 24.75 μl, and 0.25 ul Taq polymerase (QIAgen 201203). Thermal cycling was carried out using the following programme: 8 cycles of 94° C. 30 sec, 54° C. 30 sec, 72° C. for 1 min then hold at 10° C. The full length human VH-Cκ template was prepared by mixing: 5 μl 10× buffer, 10 μl 5×Q buffer, 4 μl dNTPs (2.5 mM), 1.5 μl T7A1/B (16 μM), 1.5 μl Cκ-f/F (16 μM), 2 μl of human VH-Cκ assembly products and dH₂O to 49.75 μl. 0.25 ul Taq polymerase (QIAgen 201203) was added and thermal cycling was carried out using the following programme: 30 cycles of 94° C. 30 sec, 54° C. 30 sec, 72° C. 1 min. Finally, one cycle of 72° C. for 7 min for extension, then hold at 10° C. The products of PCR were analysed by agarose gel electrophoresis (FIG. 2) and products were identified at the correct size around 700 bp as full length human VH-Cκ fragments. PCR products were taken forward directly into ribosome display selections.

### Selection on Protein A

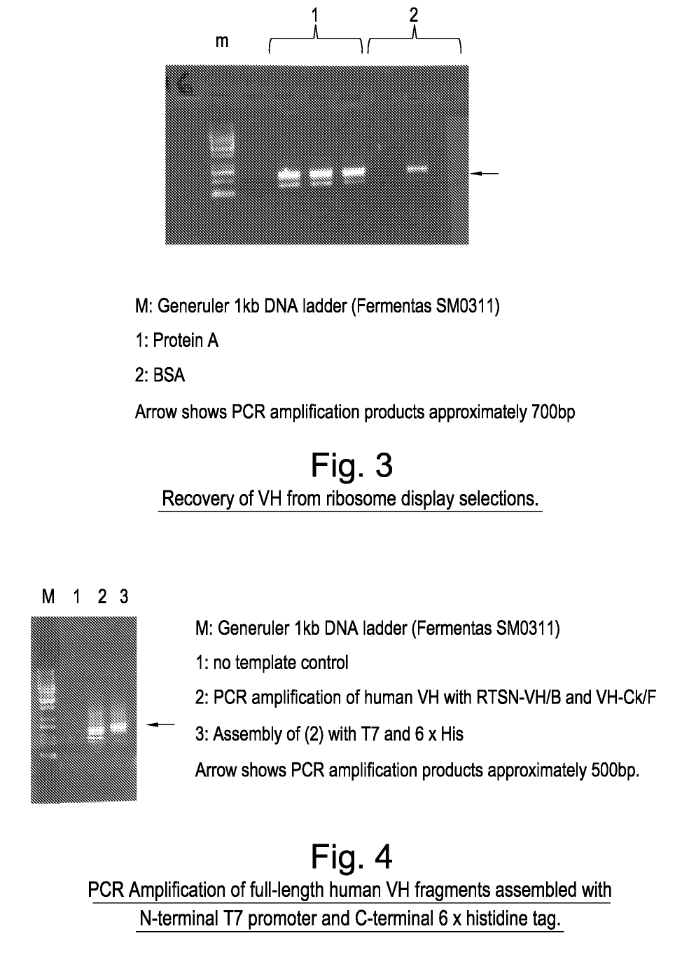

Protein A (Sigma) and BSA at 25 ug/ml in PBS were coated onto separate wells of Top Yield Strips (Nunc), 20 ul per well and incubated at 4° C. overnight. The wells were washed once with PBS and then blocked with 20 μl per well of 1% BSA in PBS for 2 hr at RT. Ribosome complexes were prepared for selection by taking the VH-Cκ PCR products into in vitro transcription-translation reactions using the TNT T7 quick kit from Promega (L1170) as follows: mixed 40 ul TNT quick solution, 0.5 ul of 0.1M magnesium acetate, 0.5 ul of methionine (1 mM—included in TNT kit), 1-3 ug of VH-Cκ PCR products and dH₂O to 50 ul final. The mix was incubated at 30° C. for 60 min after which were added 1 ul dH₂O, 7 ul of 10×DNaseI digestion buffer and 12 ul of DNaseI (Boehringer Mannheim 776-785) followed by incubation for a further 20 min at 30° C. 70 ul of 2× dilution buffer (ice-cold PBS containing 5 mM Mg acetate) was added and the transcription-translation reactions were chilled on ice. 70 μl of the TNT translation mixture, containing the PRM (protein-ribosome-mRNA) complexes was added to the protein A and BSA coated wells and incubated at 4° C. for 2 hrs. the wells were washed three times with washing buffer (PBS containing 0.01% Tween 20 and 5 mM Mg acetate) followed by 2 quick washes with 100 μl dH₂O The DNA product of bound PRM complexes was recovered by in situ RT-PCR: 12 ul of mix 1 (1 ul primer RTKz1 (16 uM), 2 ul 10 mM dNTPs and 9 ul dH₂O) were added to each well and heated to 65° C. for 5 minutes. This was placed on ice for 1 minute after which, to each well was added 8 ul of mix 2 (4 ul 5× first strand buffer, 1 ul 100 mM DTT, 1 ul dH₂O, 1 ul (20 units) RNase inhibitor (Promega N2611) and 1 ul (200 units) Superscript II enzyme (Invitrogen 18064-022)). This mix was incubated at 42° C. for 50 minutes followed by 72° C. for 15 minutes. The products were transferred to a fresh tube and then used as template in a single primer PCR reaction: 2.5 μl of 10× buffer, 5 μl of 5×Q, 2 μl of dNTP (2.5 mM), 0.75 μl Kz1 primer (16 μM), 0.5 to 1 μl cDNA (from Superscript reaction), dH₂O to 25 μl and 1 unit of Taq DNA polymerase (QIAgen 201203). Thermal cycling was carried out using the following programme: 35 cycles of 94° C. 30 sec, 48° C. 30 sec, 72° C. 1 min. Finally, one cycle of 72° C. for 7 min, then hold at 10° C. The PCR amplification products of the correct size were observed from the protein A selections, but not from the selections with BSA (as assessed by agarose gel electrophoresis; FIG. 3) The DNA from the gel was purified and used as a template for further PCR with T7A1/B and Cκ-f/F and subsequent selections by ribosome display. After 3-4 cycles of ribosome display PCR products were cloned into E. coli vectors for expression.

Expression of VH Fragments in E. coli

For expression of VH fragments, ribosome display selection outputs (PCR products) were assembled with a T7 promoter at the N-terminus and a 6× histidine tag at the C-terminus. The N-terminal T7 promoter was generated by PCR (QIAgen Taq) using the following mix: 5 μl 10× buffer, 10 μl 5×Q buffer, 4 μl dNTPs (2.5 mM), 1.5 μl RTST7/B (16 μM), 1.5 μl RTST7/F (16 μM), 10 ng of control plasmid (GFP—from Roche E. coli cell-free kit), dH₂O to 49.75 μl, and 0.25 ul Taq polymerase. The C-terminal 6× histidine tag fragment was generated by PCR using the following mix: 5 μl 10× buffer, 10 μl 5×Q buffer, 4 μl dNTPs (2.5 mM), 1.5 μl link-His/B (16 μM), 1.5 μl Tterm/F (16 μM), 10 ng of pET22b (Covagen), dH₂O to 49.75 μl, and 0.25 ul Taq polymerase. Finally, ribosome display selection outputs were amplified by PCR to generate compatible ends for assembly using the following mix: 5 μl 10× buffer, 10 μl 5×Q buffer, 4 μl dNTPs (2.5 mM), 1.5 μl RTSN-VH/B (16 μM), 1.5 μl VH-Ck/F (16 μM), 1 ul of ribosome display selection output (kz1 PCR product from protein A selection), dH₂O to 49.75 μl, and 0.25 ul Taq polymerase. For each of these PCRs 30 cycles of thermal cycling were carried out: 94° C. 30 sec; 54° C. 30 sec; 72° C. 1 min. Finally, one cycle of 72° C. for 7 min for extension, then hold at 10° C. The products of the 3 PCRs were then assembled to generate human VH fragments with a T7 promoter and C-terminal 6× histidine tag using the following mix: 2.5 μl 10× buffer, 5 μl 5×Q buffer, 1 μl dNTPs (2.5 mM), 10-50 ng of gel purified T7 promoter fragment, 10-50 ng of gel purified ribosome display PCR products (RTSN-VH/B and VH-Ck/F primers), 10-50 ng of gel purified C-terminal 6× histidine tag fragment, dH₂O to 24.75 μl, and 0.25 ul Taq polymerase (QIAgen 201203). 8 cycles of thermal cycling were carried out: 94° C. 30 sec; 54° C. 30 sec; 72° C. 1 min. Finally, hold at 10° C. Full length T7-VH-6×His were prepared using the following mix: 5 μl 10× buffer, 10 μl 5×Q buffer, 4 μl dNTPs (2.5 mM), 1.5 μl RTST7/B (16 μM), 1.5 μl Tterm/F (16 μM), 41 of human T7-VH-6×His assembly products and dH₂O to 49.75 μl. 0.25 ul Taq polymerase (QIAgen 201203) was added and thermal cycling carried out as follows: 94° C. 30 sec; 54° C. 30 sec; 72° C. 1 min. Finally, one cycle of 72° C. for 7 min for extension, then hold at 10° C. PCR products were analysed by agarose gel electrophoresis (FIG. 4) and material of around 500 bp cloned directly into TA vectors (Invitrogen) following the manufacturer's instructions. The ligation products were chemically transformed into E. coli strain JM109 (DE3) using the KCM method (Chung & Miller, 1988, Nucleic Acids Res.; 16:3580).

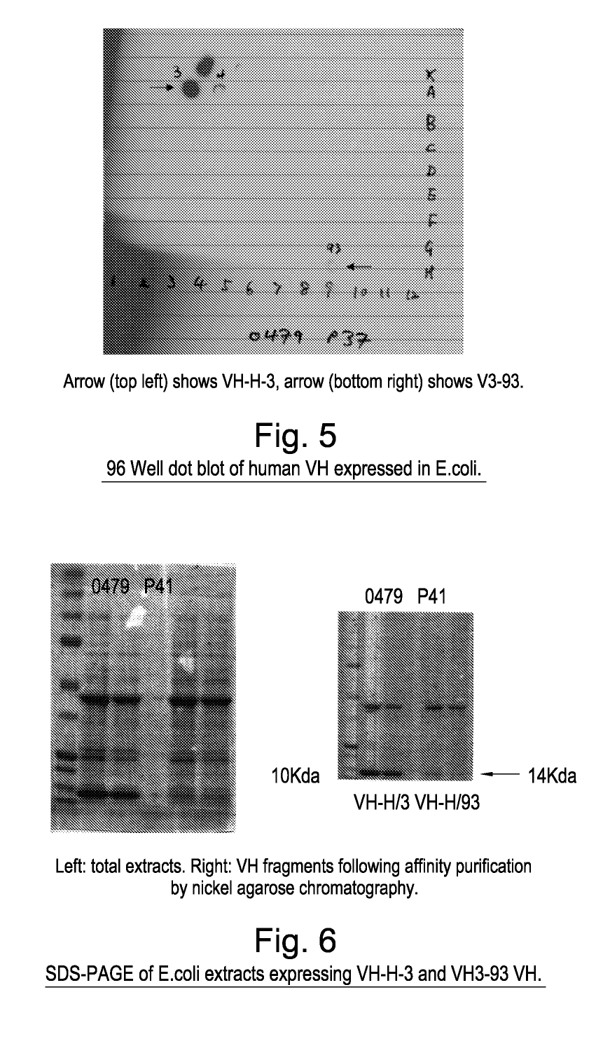

Individual colonies from the ligation and transformation were picked into 96-well deep well plates (Nunc) containing 1 ml/well of L-broth supplemented with 100 ug/ml ampicillin and 1% (w/v) glucose. Plates were grown overnight at 37° C. with shaking at 250 rpm. Plates were then centrifuged at 4000×g for 15 minutes and the supernatant discarded. Cell pellets were resuspended with 1 ml per well of 2×TY medium supplemented with 100 ug/ml ampicillin and 1 mM IPTG and plates incubated at 30° C. with shaking at 250 rpm for 3 to 5 hours. Plates were then centrifuged at 4000×g for 15 minutes and the supernatant discarded. VH fragments were extracted from the cell pellets by adding 150 ul BugBuster (Novagen) to each well and resuspending the cell pellets by pipetting. Extracts were transferred to eppendorf tubes and centrifuged for 20 minutes at 13000 rpm. 5 ul of each extract was spotted onto an Immobilon-P membrane (Millipore), after which the membrane was dried briefly then blocked with 1% BSA. Soluble VH fragments were detected using anti-His-HRP conjugate antibody (Sigma A7058) diluted 1:4000 and blots developed by ECL (Perbio 34080) (FIG. 5). Sequencing of positive/soluble VH fragments from these blots identified clones VH-H-3 and VH3-93, each of which were subsequently grown up again as described and expression scaled up to 25 ml cultures. VH fragments were purified from Bugbuster extracts by nickel agarose affinity chromatography and analysed by SDS-PAGE (FIG. 6). Other VH fragment sequences isolated from this dot-blot approach were: (clone names) 3rdPAVH1-70, 3rdPAVH2-51, 3rdPA-VH-85, 3rdPAVH2-16, 3rdPA-VH-93, 3rdPA-VH-91, VH1-3 and VH5-5.

### Example 2: Methods for Preparation of CDR3 Domains

Human cDNA from spleen, lymph node, bone marrow and peripheral blood lymphocytes was purchased from commercial sources (Invitrogen, Clontech). Oligonucleotide primers VHCDR3/B and VHJ/F were synthesised to facilitate PCR amplification of VH-CDR3 plus VH framework 4 sequences from B cell cDNA.

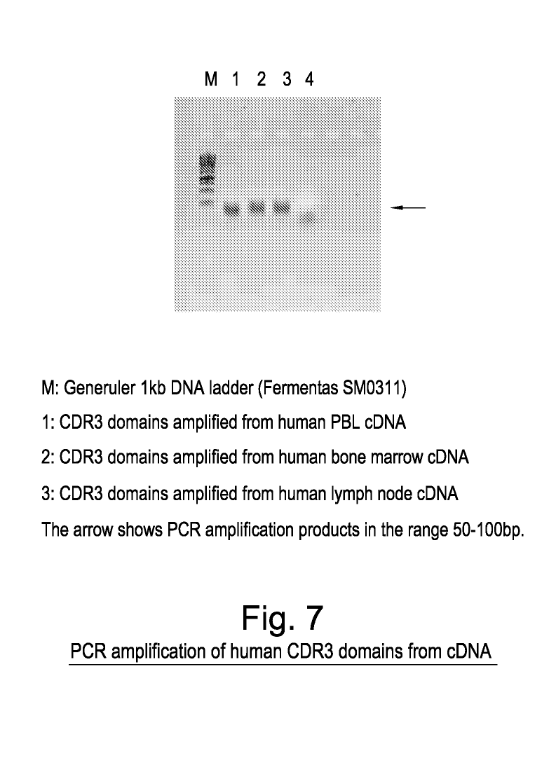

Individual PCR reactions were set up for each cDNA sample as follows: 25 ul 2×Phusion PCR mix (Finnzymes F-531L); 2.5 ul VHCDR3/B (10 uM); 2.5 ul VHJ/F (10 uM); 3 ng cDNA and dH₂O to 50 ul final. Reactions were then heated to 95° C. for 1 minute followed by 30 cycles of PCR: 98° C. 10 seconds, 54° C. 30 seconds, 72° C. 30 seconds. After 30 cycles PCR reactions were then heated at 72° C. for 8 minutes followed by holding at 10° C. PCR products were then analysed by electrophoresis on 1% (w/v) agarose gels followed by staining with ethidium bromide. PCR amplification products were observed at the correct size (approximately 50-100 bp; FIG. 7).

### Example 3: Library Assembly

The VH-H-3 scaffold was amplified by PCR (QIAgen Taq 201203) using the following mix: 5 μl 10× buffer, 10 μl 5×Q buffer, 4 μl dNTPs (2.5 mM), 1.5 μl T7AB/VH3 (16 μM), 1.5 μl VHJ/F (16 μM), 10 ng of plasmid encoding VH-H-3 were mixed and dH₂O added to 49.75 μl followed by 0.25 ul Taq polymerase. The VH3-93 scaffold was amplified by PCR in the same way, replacing primer T7AB/VH3 with VH3-93/B and using a plasmid encoding VH3-93. For both PCRs 30 cycles of thermal cycling were carried out: 94° C. 30 sec; 54° C. 30 sec; 72° C. 1 min. Finally, one cycle of 72° C. for 7 min for extension, then hold at 10° C.

Human VH-CDR3 PCR products (Example 2) were then assembled with either VH-H-3 or VH3-93 scaffolds to generate DNA products encoding full length VH antibodies. VH-H-3 or VH3-93 scaffolds were assembled with amplified human VH-CDR3 sequences in separate PCR reactions by adding the following: 12.5 ul 2× Phusion PCR mix (Finnzymes F-531L); 10 ng of either VH-H-3 or VH3-93 PCR products; 40 ng of each VH-CDR3 PCR product (Example 2) and dH₂O to 25 ul final. Reactions were then heated to 95° C. for 1 minute followed by 8 cycles of PCR: 98° C. 10 seconds, 54° C. 30 seconds, 72° C. 30 seconds. After 8 cycles, PCR reactions were then heated at 72° C. for 8 minutes followed by holding at 10° C.

Full-length VH products were then amplified from the assembly products by pull-through PCR using the following reaction conditions:
(a) For the VH-H-3 scaffold: 25 ul 2× Phusion PCR mix (Finnzymes F-531L); 2.5 ul of oligonucleotide T7AB/VH3 (10 uM); 2.5 ul of oligonucleotide VHJ/F (10 uM); 5 ul of VH-H-3 assembly products and dH₂O to 50 ul final volume.
(b) For the VH3-93 scaffold: 25 ul 2× Phusion PCR mix (Finnzymes F-531L); 2.5 ul of oligonucleotide VH3-93/B (10 uM); 2.5 ul of oligonucleotide VHJ/F (10 uM); 5 ul of VH3-93 assembly products and dH₂O to 50 ul final volume.

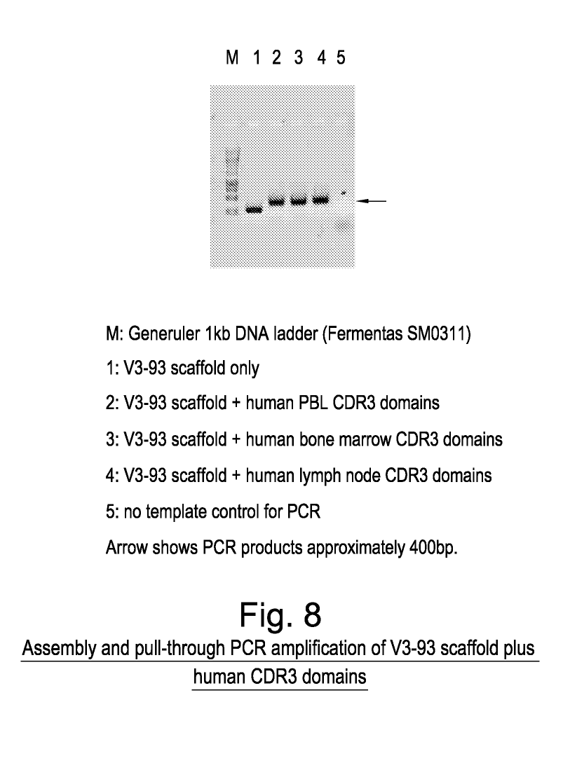

Reactions were then heated to 95° C. for 1 minute followed by 30 cycles of PCR: 98° C. 10 seconds, 54° C. 30 seconds, 72° C. 30 seconds. After 30 cycles PCR reactions were then heated at 72° C. for 8 minutes followed by holding at 10° C. Products of PCR were then analysed by electrophoresis on 1% (w/v) agarose gels followed by staining with ethidium bromide. Full length VH products were observed at the expected size of approximately 400 bp (FIG. 8). The PCR products were purified using Fermentas PCR purification columns (K0701) and resuspended in dH₂O.

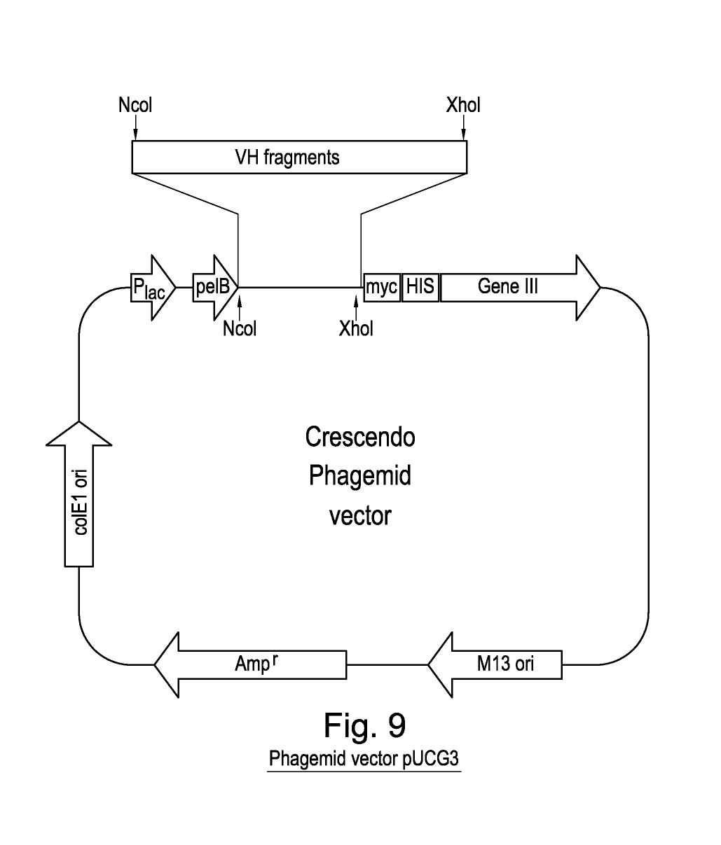

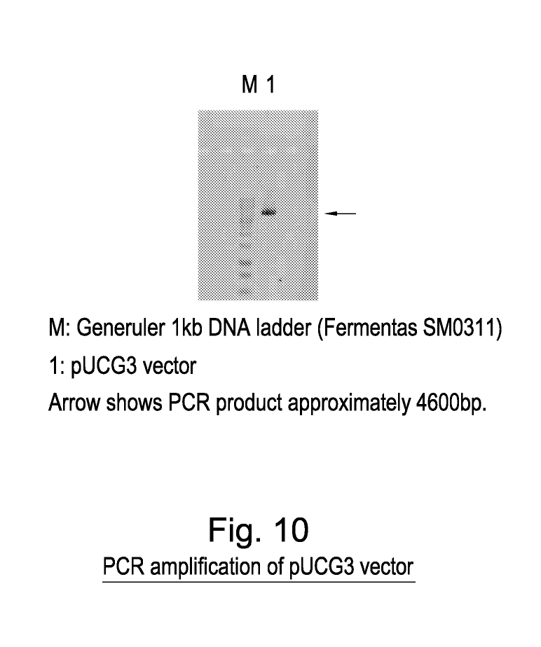

To prepare libraries for phage display, full-length VH products were cloned into phagemid vector pUCG3 (FIG. 9). Phagemid DNA for cloning was prepared by PCR as follows: 1000 ul 2× Phusion PCR mix (Finnzymes F-531L); 60 ul of oligonucleotide pHENAPmut4 (16 uM); 60 ul of oligonucleotide pHENAPmut5 (16 uM); 400 ng of pUCG3 miniprep DNA and dH₂O to 2000 ul final volume. The reaction was divided equally into 40 tubes and then heated to 95° C. for 1 minute followed by 30 cycles of PCR: 98° C. 10 seconds, 72° C. 2 minutes. After 30 cycles the PCR reactions were then heated at 72° C. for 5 minutes followed by holding at 10° C. Products of PCR were then analysed by electrophoresis on 1% (w/v) agarose gels followed by staining with ethidium bromide. PCR products were observed at the expected size of approximately 4600 bp (FIG. 10). The PCR product was purified using Fermentas PCR purification columns (K0701) and resuspended in dH₂O.

Both the pUCG3 vector preparation and VH-H-3/VH3-93 PCR products were digested with NcoI (Fermentas FD0574) and XhoI (Fermentas FD0694) restriction enzymes overnight at 37° C. The pUCG3 restriction digest only was then incubated with shrimp alkaline phosphatase for 4 hours at 37° C. according to the manufacturers instructions (Fermentas EF0511). All digests were heated to 80° C. for 5 minutes and then each product purified using Fermentas PCR purification columns (K0701) and finally resuspended in dH₂O.

The digested VH products were ligated into pUCG3 using NEB T4 DNA ligase (M0202M) following the manufacturers instructions. Briefly, NcoI/XhoI double-digested pUCG3 DNA and VH products were mixed at a molar ratio of 1:2 and incubated overnight with T4 ligase at 16° C. Following incubation at 70° C. for 30 minutes, the products of ligation were purified using Fermentas PCR purification columns and finally resuspended in dH₂O. Then, using Biorad cuvettes (165-2089) and a Biorad Micropulser, 2 ul of the purified ligation products were electroporated into 25 ul of electrocompetent TG1 cells (Lucigen 60502-1) following the manufacturer's instructions. Electroporated TG1 cells were plated onto 2×TY agar plates supplemented with ampicillin at 100 ug/ml and glucose at 20% (w/v) and incubated overnight at 30° C. Also a dilution series of electroporated TG1 cells were plated to determine library size. The library sizes were calculated as 1×10⁹ recombinants for the VH-H-3 spleen library and 8×10⁹ for the VH3-93 library. Successful library construction was confirmed by sequence analysis revealing that 94% of VH possessed unique CDR3 sequences of between 5 and 26 amino acids in length.

### Example 4: Analysis of Library Composition to Determine the Proportion of Soluble Clones

The solubility of VH fragments produced from each library was investigated by analysis of bacterial periplasmic extracts. All VH fragments include at their N-terminus a pelb leader sequence that directs them to the periplasmic space following expression. Thus, VH fragments that are insoluble or aggregated accumulate in the cytoplasm as inclusion bodies and only soluble VH is able to cross the bacterial membrane into the periplasm. Therefore, detection of VH fragments in bacterial periplasmic extracts is a good surrogate measure of VH solubility and an ELISA-based method was developed for this purpose.

Over 90 individual colonies from each library were picked into wells of a Nunc 96 deep well plate containing 1000 ul per well of 2×YT broth supplemented with 2% (w/v) glucose and 100 ug/ml ampicillin. The plates were then grown at 37° C. with shaking at 250 rpm for 5-6 hours. Plates were centrifuged at 3200 rpm for 10 mins and the supernatant discarded. Cell pellets were then resuspended in 1 ml 2×YT containing 100 ug/ml ampicillin and 1 mM IPTG, and the plates incubated overnight at 30° C. with shaking at 250 rpm. Plates were centrifuged at 3200 rpm for 10 mins and the cell pellets resuspended in 80 ul of sucrose buffer (20% sucrose, Babraham Stores 101361, 1 mM EDTA, Sigma E5134, 50 mM Tris-HCl pH 8, Melford 1185-53-1), and then placed on ice for 30 mins. The plates were then centrifuged at 4500 rpm for 15 mins and 50 ul of supernatant from each well transferred to the corresponding well of a Nunc 96 well maxisorb plate (Nunc 443404). This supernatant, the bacterial periplasmic extract (containing any soluble expressed VH), was then incubated for 2 hours at room temperature to coat proteins onto the plate.

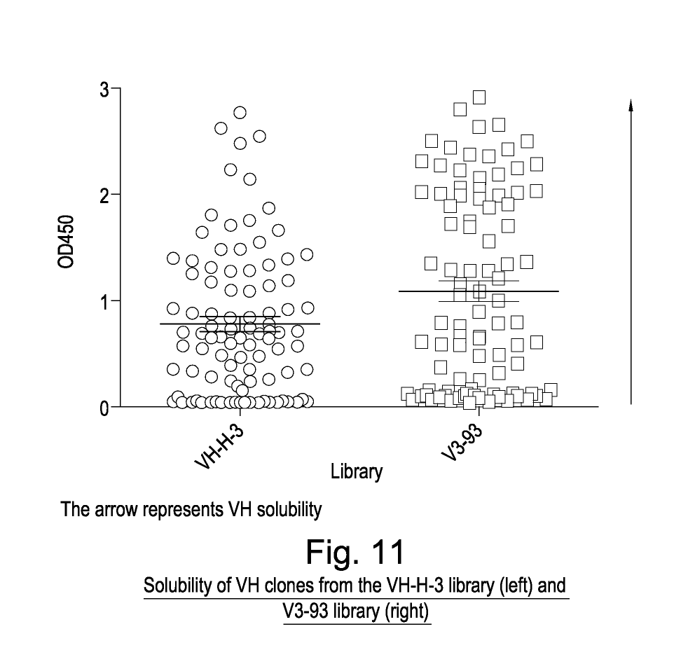

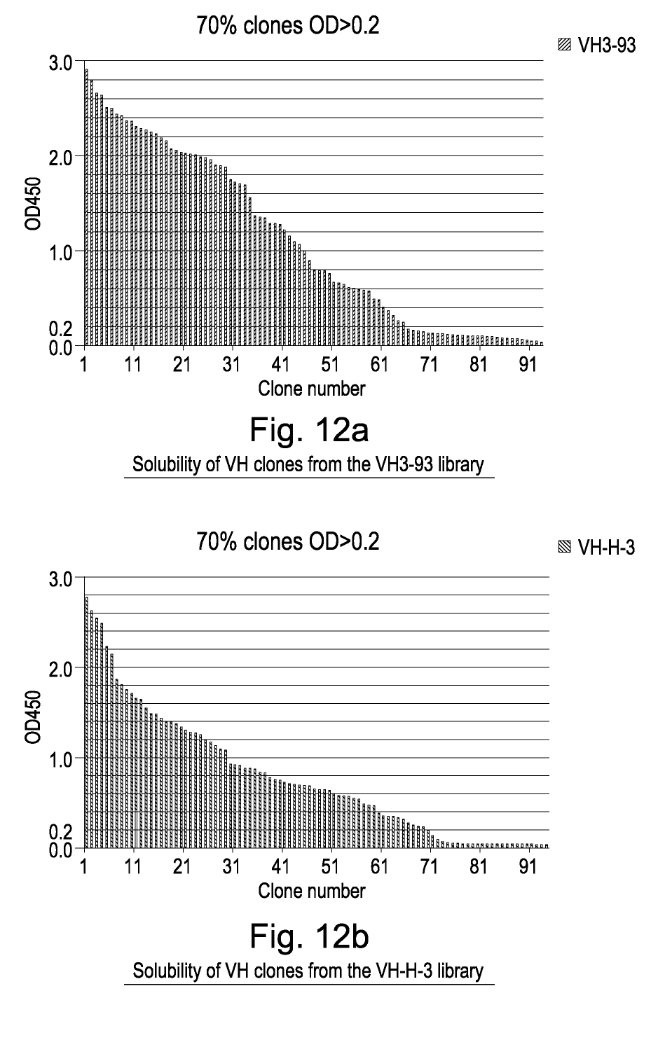

The wells of the Nunc plates were then washed once with PBS buffer and then 3% (w/v) Marvel in PBS was added (200 ul per well). Plates were then incubated for 1 hour at room temperature. The wells of the Nunc plates were again washed once with PBS buffer and then 50 ul per well of HRP-conjugated anti-HIS monoclonal antibody (Miltenyi Biotech, 130-092-7853%), diluted 1:1000 in 3% (w/v) Marvel PBS added. Plates were then incubated for a further 1 hour at room temperature. The wells of the Nunc plates were then washed three times with PBST buffer followed by three washes with PBS buffer, and then to each well was added 50 ul of TMB developer (Sigma T0440). Plates were incubated for up to 10 minutes and then TMB development was stopped by the addition of 25 ul per well of 0.5M sulphuric acid solution. Plates were then read on a Biorad iMark plate reader to measure the absorbance at 450 nm in each well. Solubility results were then plotted on graphs (FIG. 11). The percentage of clones having an OD of 0.2 and above was found to be at least 70% for both libraries (FIGS. 12a and 12b).

### Example 5: Screening Libraries Against Antigen

The VH-H-3 and VH3-93 libraries were used to generate VH antibodies to protein antigens by phage display. Preparation of library phage stocks and phage display selections were performed according to published methods (Antibody Engineering, Edited by Benny Lo, chapter 8, p 161-176, 2004). Selections were performed on 4 different protein antigens: TNF-α (Gift from Andreas Hoffmann, Martin-Luther-Universitat Halle-Wittenberg), KLH (Merck 374825), human ovalbumin (Sigma A5503) and human TNFR1 (Sino 10872). All antigens were immobilised onto maxisorb plates (Nunc 443404) at 10 ug/ml in PBS and two rounds of phage display selection were performed.

### Example 6: Analysis of Isolated VH Domains and Sequencing

Following selections of the VH-H-3 and VH3-93 libraries on TNF-α, KLH, human ovalbumin and human TNFR1, VH antibodies specific for each antigen were identified by phage ELISA following published methods (Antibody Engineering, Edited by Benny Lo, chapter 8, p 161-176, 2004). For each selection, phage ELISAs were performed against the target antigen and an unrelated antigen as control. DNA sequencing of VH clones shown to bind specifically to antigen was performed to analyse diversity of VH produced to each antigen (Table 2).

TABLE 2
Summary of VH isolated to ovalbumin, TNF-a, TNFR1 and KLH

| | Colonies | Specific V_{H} by | Number | |
|---|---|---|---|---|
| Antigen | picked | ELISA | sequenced | Unique V_{H} |
| TNF-R1 | 464 | 368 | 103 | 30 |
| TNF-α | 1307 | 423 | 423 | 63 |
| Ovalbumin | 96 | 22 | 22 | 1 |
| KLH | 92 | 47 | 26 | 14 |

A number of clones were sequenced for each antigen (Table 3) and the output was found have expected levels of diversity.

TABLE 3
CDR3 sequences of VH isolated to
ovalbumin, KLH, TNFR1 and TNF-α

| | VH | | Seq ID |
|---|---|---|---|
| Antigen | scaffold | CDR3 sequence | No |
| Ovalbumin | VH-H-3 | PAGYDAFDI | 26 |
| KLH | VH-H-3 | DRGSSISDPFDI | 27 |
| | | EAPWLAQYDAFDI | 28 |
| | | GQDGYDGFDI | 29 |
| | | PSELSGWFSP | 30 |
| | V3-93 | DKWDDIKQFDN | 31 |
| | | DSDVDMYGYYTFES | 32 |
| | | EASYYDTTGYKIFDL | 33 |
| | | EMDYDKVGYSQFDY | 34 |
| | | EPGRYYFDGSDYEDV | 35 |
| | | ESPYNDDHYIMDS | 36 |
| | | EVEYGGGLYDFDV | 37 |
| | | KWNDVDS | 38 |
| | | QWNNWHPN | 39 |
| TNFR1 | VH-H-3 | DAQI | 40 |
| | | DEDI | 41 |
| | | DEDT | 42 |
| | | DEPPGAFDI | 43 |
| | | DGAAAGLDAFDI | 44 |
| | | DKDI | 45 |
| | | DKDY | 46 |
| | | DKHI | 47 |
| | | DMQQ | 48 |
| | | DNMAFDI | 49 |
| | | DQDY | 50 |
| | | DSSGWPFDY | 51 |
| | | EDGTIGAFDI | 52 |
| | | EDLESSGEDS | 53 |
| | | EDYGDAFDI | 54 |
| | | EGSGSRYAFDI | 55 |
| | | EGYGDAFDI | 56 |
| | | EIGI | 57 |
| | | EIQTGDDY | 58 |
| | | EKDYGMDV | 59 |
| | | ELAGAFDI | 60 |
| | | ENRDGEDV | 61 |
| | | ENSYDTDV | 62 |
| | | ETQTGDDY | 63 |
| | | EWPLAGPDAFDI | 64 |
| | | EYDYGMDV | 65 |
| | | EYDYGTDV | 66 |
| | | EYHYGEDV | 67 |
| | | FIRGNWLPDAFDL | 68 |
| | | GPSHGGFDI | 69 |
| | | GRRGWSAFDI | 70 |
| | | NEDV | 71 |
| | | SFYIEGRTRAFGI | 72 |
| | V3-93 | DKDN | 73 |
| | | DKDV | 74 |
| | | EARGGGYSMGYGSFDY | 75 |
| | | EDDFQNSYYVDV | 76 |
| | | EDNFEDSYYVDV | 77 |
| | | EDWNLGRGMDV | 78 |
| | | EDYGDSQYLEALDV | 79 |
| | | EPYDDYDSDSMDV | 80 |
| | | ERPGREFYGMDV | 81 |
| | | ESDMGDV | 82 |
| | | GKTAAAGGFDN | 83 |
| | | GLYRHGQGLDP | 84 |
| | | GMYNWNDRNALDI | 85 |
| | | GPHDSSYYYGLDV | 86 |
| | | GTQRQLSP | 87 |
| | | GYFDWLAPPVV | 88 |
| | | LHHDFWSVDDTFDV | 89 |
| | | QNCGSPDCSYGGFDP | 90 |
| | | RFFDWLQGSRYYGMDV | 91 |
| TNF-α | VH-H-3 | AARGTRELSTVDV | 92 |
| | | AQTSGIYTYYYHTMDV | 93 |
| | | ASVGSRPHTFDI | 94 |
| | | DAGFGTGLSLRYYHYMDV | 95 |
| | | DDILTGRMDV | 96 |
| | | DFGDYGHSGFDM | 97 |
| | | DGGSGSLMHDAFDI | 98 |
| | | DHGDYYYYHSMDV | 99 |
| | | DIRLPASMRDDFFYFGMDV | 100 |
| | | DLFDLWSGYFHDAFDI | 101 |
| | | DLGHDFWSGYYHDAFDI | 102 |
| | | DPRKVAPRAFDI | 103 |
| | | DPVAGTSVPSGFDL | 104 |
| | | DPYSGRYGNEHYHYMDV | 105 |
| | | DPYSGRYGNEYYHYMDV | 106 |
| | | DRFLQRTWSRPHDAFDM | 107 |
| | | DSGYNAFDI | 108 |
| | | DSRGGGSYPYYHGMDV | 109 |
| | | DVYSSGRSFDY | 110 |
| | | DWGSHYCDSMGPRRPRKAFDI | 111 |
| | | DWGSYYHDSSDPRRPHEAFDL | 112 |
| | | DWGSYYHDSSGPRRPHEAFDI | 113 |
| | | DWGSYYYDSSGPRRPHEAFDI | 114 |
| | | EGQYLWLPRHYYHGLDV | 115 |
| | | EWVLGDKSVFDV | 116 |
| | | EYCRSETCLMDV | 117 |
| | | GAGYCSGGSCYPGGVFDI | 118 |
| | | GDFWSGAWHDAFDI | 119 |
| | | GDGYCSGGSCYPGGAFDI | 120 |
| | | GFWSGYLHDAFDI | 121 |
| | | GGSGHGSYYYFHTMDV | 122 |
| | | GGSGWYLSNAFDI | 123 |
| | | GGYCSSTSCLVHTFDI | 124 |
| | | GIAAVTKDYNYYYHAMDV | 125 |
| | | GIATVTKDYNYYYHAMDV | 126 |
| | | GISATDYYYHGMDV | 127 |
| | | GLERGDVFHHFDY | 128 |
| | | GLIDGDYYYHGMDV | 129 |
| | | GLPTDRAFDV | 130 |
| | | GLSGPQWHYYHYMDV | 131 |
| | | GPDYGGNGPVGAFDI | 132 |
| | | GPEGSSSFLGAFDI | 133 |
| | | GRIRDGYFHDAFDI | 134 |
| | | GSGRYYYHGMDV | 135 |
| | | GSGSWAFDI | 136 |
| | | GSVGTRPHTFDI | 137 |
| | | GSVGTRPHTFDV | 138 |
| | | GTAHSYYHLMDV | 139 |
| | | GTEYYYHDMDV | 140 |
| | | GTLVPTGHYHTLDV | 141 |
| | | GVAYSYYHHMDV | 142 |
| | | GVTSAFVFAFDI | 143 |
| | | GVTSAFVFAFDV | 144 |
| | | GVVGSRPHTFDI | 145 |
| | | GVVPAGHYYHYMDV | 146 |
| | | GWELGLDD | 147 |
| | | GWGSYFHAFDI | 148 |
| | | GWYASDI | 149 |
| | | GYYDMDV | 150 |
| | | HEALMTTWLLDV | 151 |
| | | HPGELGAFDI | 152 |
| | | HSDARWPPNFDY | 153 |
| | | NLGHDFWSGYYHDAFDI | 154 |
| | | QEGLVDSYYGMDV | 155 |
| | | RFRYSSSSDVFDI | 156 |
| | | RFWYSSSSDVFDI | 157 |
| | | RGSGHGSYYYFHTMDV | 158 |
| | | RHDSGKYRYHDAFDI | 159 |
| | | RHESLNAFDV | 160 |
| | | RHLLLDVFDV | 161 |
| | | RSGYGSGPVYYYHYGMDV | 162 |
| | | RSYYSSSLQREIHYGMDV | 163 |
| | | SAEHWVAPNYYFHNMDV | 164 |
| | | TESSGSSPYYYHYMDV | 165 |
| | | TTGKQQLPRGAFDI | 166 |
| | | VDTLTKAFDV | 167 |
| | | VFRYSSSSDVFDI | 168 |
| | | VRSGPYDPFDI | 169 |
| | | WIQPFDY | 170 |
| | | WLQPFDY | 171 |
| | | YGVVGGRRYFDY | 172 |

### Example 7: Analysis of VH Solubility, Expression, Stability and Aggregation

VH antibodies from selections on KLH, ovalbumin and TNFR1 from both libraries were expressed and purified from 50 ml shake flask cultures. Each VH protein has a C-terminal 6×HIS tag that enables purification from bacterial perisplamic extracts by nickel-agarose affinity chromatography.

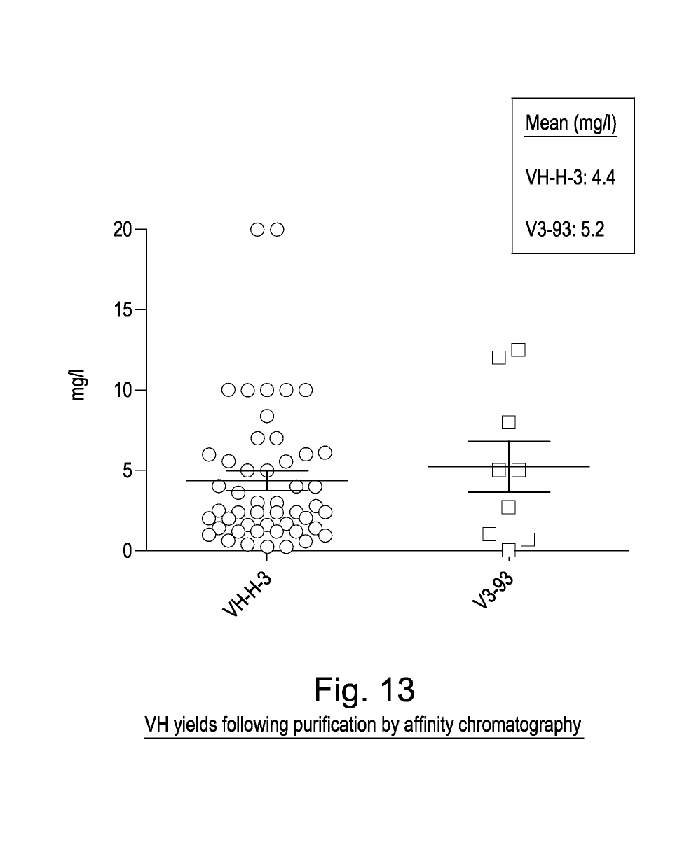

A starter culture of each VH was grown overnight in 5 ml 2×TY broth (Melford, M2103) supplemented with 2% (w/v) glucose+100 ug/ml ampicillin at 30° C. with 250 rpm shaking. 50 ul of this overnight culture was then used to inoculate 50 ml 2×TY supplemented with 2% (w/v) glucose+100 ug/ml ampicillin and incubated at 37° C. with 250 rpm shaking for approximately 6-8 hours (until OD600=0.6-1.0). Cultures were then centrifuged at 3200 rpm for 10 mins and the cell pellets resuspended in 50 ml fresh 2×TY broth containing 100 ug/ml ampicillin+1 mM IPTG. Shake flasks were then incubated overnight at 30° C. and 250 rpm. Cultures were again centrifuged at 3200 rpm for 10 mins and supernatants discarded. Cell pellets were resuspended in 1 ml ice cold extraction buffer (20% (w/v) sucrose, 1 mM EDTA & 50 mM Tris-HCl pH8.0) by gently pipetting and then a further 1.5 ml of 1:5 diluted ice cold extraction buffer added. Cells were incubated on ice for 30 minutes and then centrifuged at 4500 rpm for 15 mins at 4° C. Supernatants were transferred to 50 ml Falcon tubes containing imidazole (Sigma, I2399—final concentration 10 mM) and 0.5 ml of nickel agarose beads (Qiagen, Ni-NTA 50% soln, 30210) pre-equilibrated with PBS buffer. VH binding to the nickel agarose beads was allowed to proceed for 2 hours at 4° C. with gentle shaking. The nickel agarose beads were then transferred to a polyprep column (BioRad, 731-1550) and the supernatant discarded by gravity flow. The columns were then washed 3 times with 5 ml of PBS+0.05% Tween followed by 3 washes with 5 ml of PBS containing imidazole at a concentration of 20 mM. VH were then eluted from the columns by the addition of 250 ul of PBS containing imidazole at a concentration of 250 mM. Imidazole was then removed from the purified VH preparations by buffer exchange with NAP-5 columns (GE Healthcare, 17-0853-01) and then eluting with 1 ml of HBS-EP buffer (Biacore, BR-1006-60). Yields of purified VH from the VH-H-3 and VH3-93 libraries are summarised in FIG. 13.

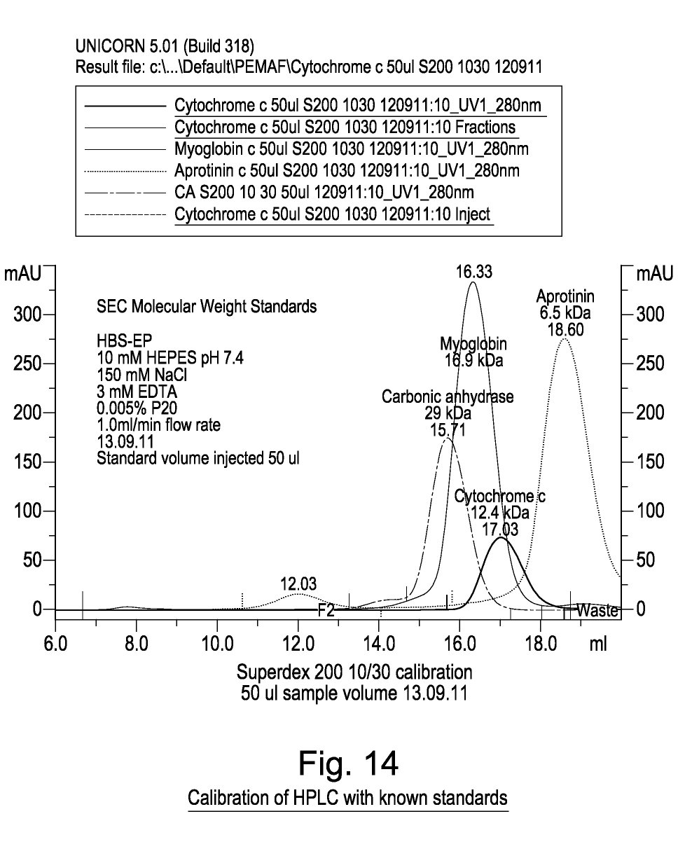

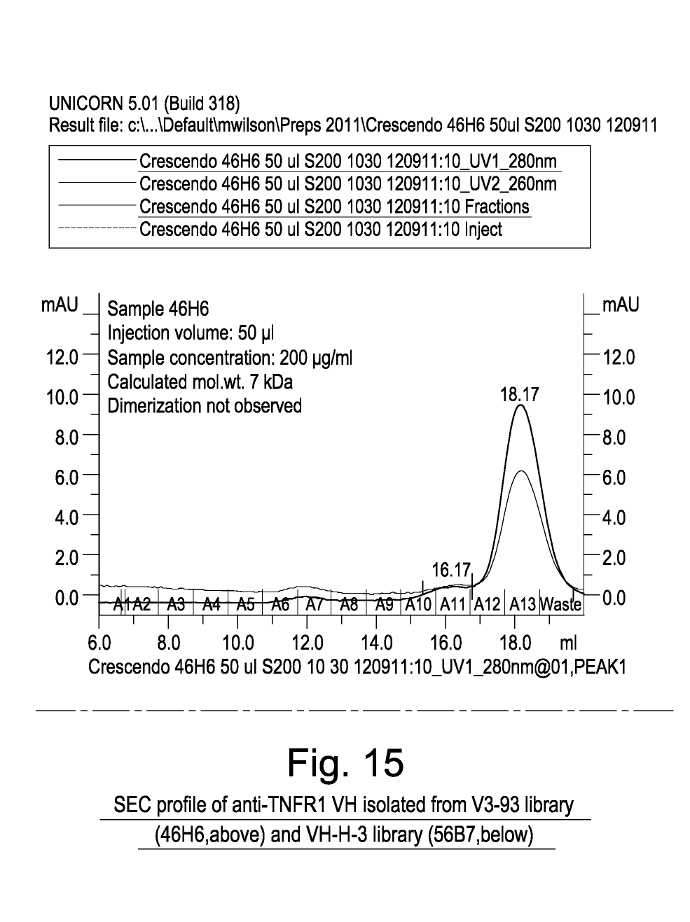

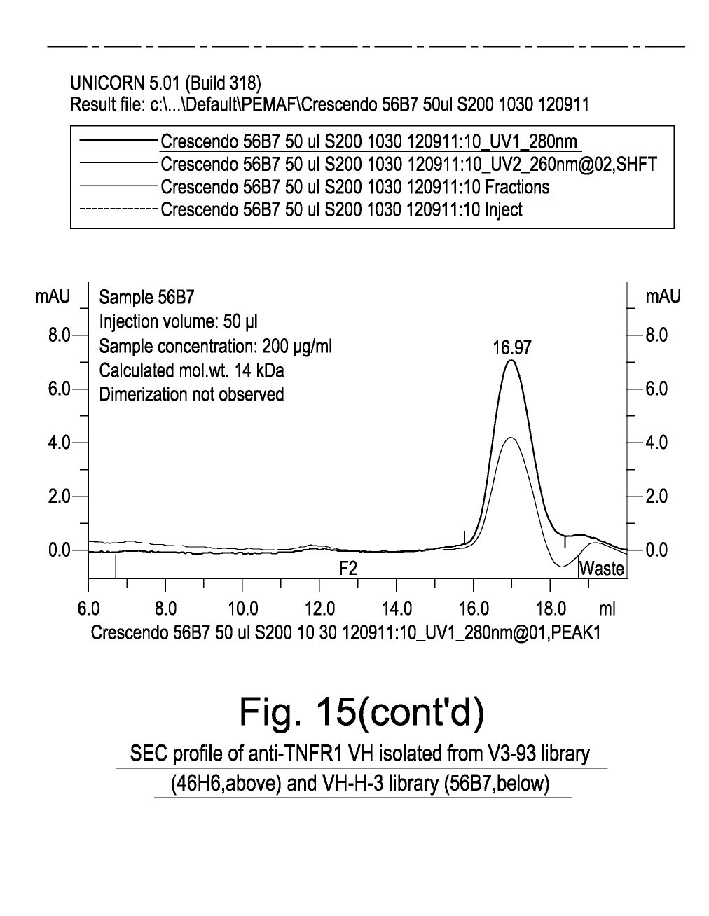

VH stability and aggregation was determined by SEC (size exclusion chromatography) using the Äkta Explorer FPLC and a Superdex 200 10/30 HR column (GE lifesciences). VH samples were diluted to 200 ug/ml in HBS-EP buffer and centrifuged at 18000×g for 10 min 4° C. 50 ul of VH was then injected onto the Superdex column and elution monitored by absorbance at 280 nm. Molecular weights were determined by comparison with the elution profiles of known standards (FIG. 14). SEC traces for two anti-TNFR1 VH (46H6 from V3-93 and 56B7 from VH-H-3) are presented in FIG. 15.

### Example 8: Anti-TNFR1 VH Inhibit Binding of TNF-α to TNFR1 in a Competition Binding Assay

To demonstrate whether anti-TNFR1 VH possessed inhibitory properties, a binding assay was developed to measure binding of TNF-α to TNFR1. Inhibitory VH would, on binding TNFR1, block TNF-α and thus reduce the signal observed in the assay.

TNFR1 (Sino Biologics, 10872-H03H) was diluted to 0.2 ug/ml (1.8 nM) in PBS and 50 ul per well added to a Nunc maxisorp 96 well plate (Fisher, DIS-071-010P). The plate was then incubated overnight at 4° C. The plate was washed once in PBS, 200 ul per well of blocking buffer (3% marvel in PBS) added and then incubated for 1 hour at room temperature. Dilution series of anti-TNFR1 VH were prepared in blocking buffer and incubated for 1 hour at room temperature in Greiner plates (650207). The TNFR1 coated maxisorp plate was then washed once with PBS and 40 ul per well of each VH dilution series transferred from the Greiner plate to the corresponding wells of the maxisorp plate. Following incubation for 1 hour at room temperature, 10 ul per well of biotinylated-TNF-α (Gift from Andreas Hoffmann, Martin-Luther-Universität Halle-Wittenberg) was added to a final concentration of 1 nM and the plate incubated for 1 hour at room temperature. The plate was washed 3 times with PBS Tween and then 3 times with PBS and then 50 ul per well of Neutravidin-HRP (Pierce, 31030) added at a dilution of 1:5000 in blocking buffer. The plate was again incubated for 1 hour at room temperature following which it was washed 3 times with PBS Tween and then 3 times with PBS. Then 50 ul of TMB developer solution (Sigma T0440) was added to each well and the plate allowed to incubate at room temperature until suitable blue colour had developed. Then 50 ul of 0.5M sulphuric acid was added to each well to stop the reaction and absorbance at 450 nm read on a spectrophotometer.

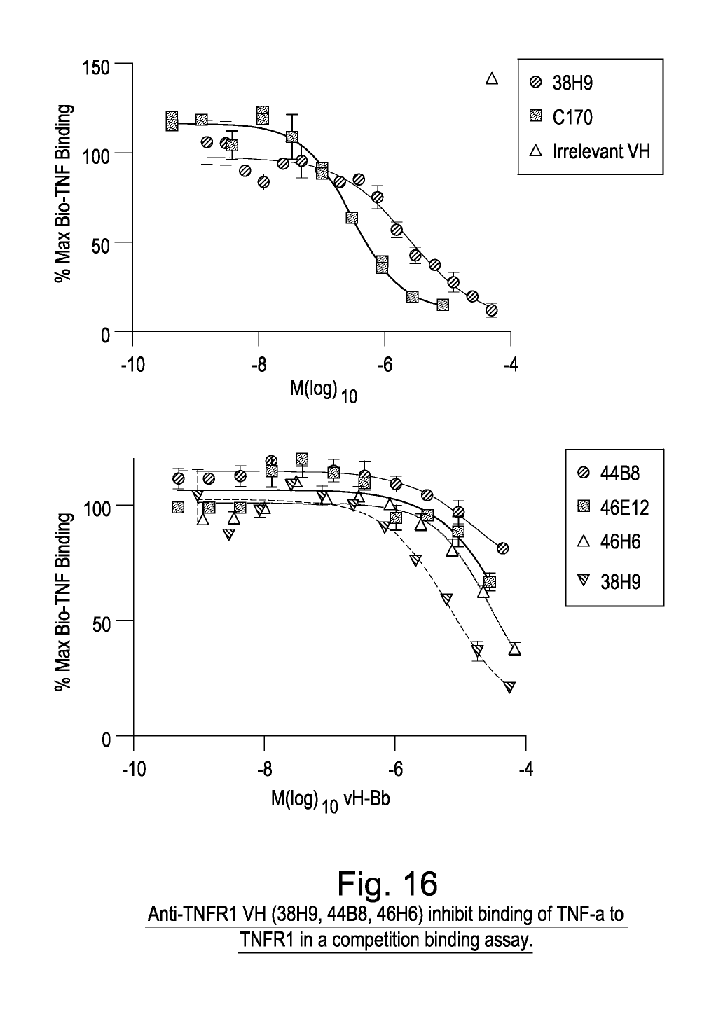

The activity of several anti-TNFR1 VH were measured in this assay and several candidates with inhibitory properties were identified (38H9, 44B8, 46E12, 46H6, FIG. 16). 38H9 was derived from library VH-H-3, the remaining clones were derived from library VH3-93. The identification of anti-TNFR1 VH antibodies as described herein, with high affinity, antigen specificity and which are also soluble and stable, validates the utility of libraries derived from the scaffolds of the invention in the isolation of further VH antibodies to other target antigens with comparable solubility, functionality and stability characteristics.

### Example 9: Generation of 81G1, a VH3 Heavy Chain Only Antibody Lacking Protein A Binding Activity

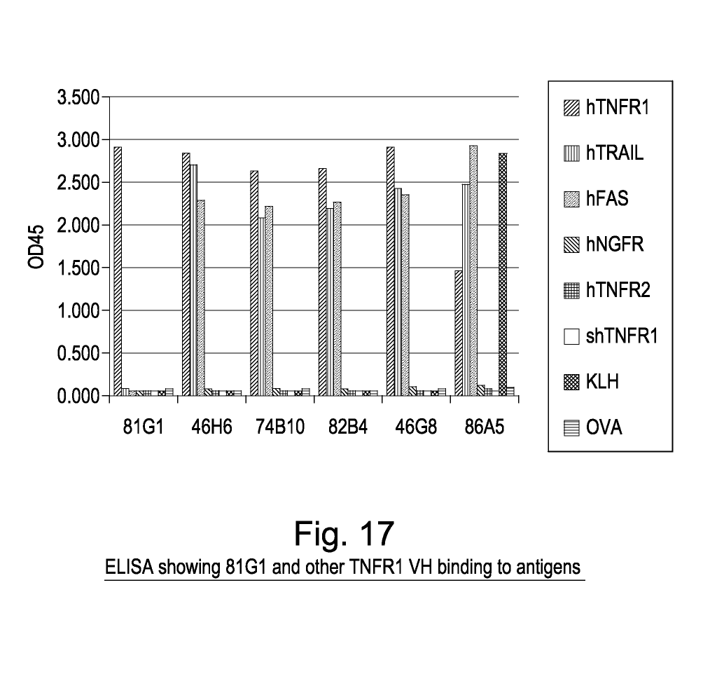

The presence of protein A in preparations of TNFR1, TRAIL and Fas gives the false impression of binding by several human VH3 antibodies due to the presence of residual protein A (FIG. 17). Here we describe the identification of a single amino acid change in VH3 FR3 that disrupts the protein A binding site and does not affect VH functionality with respect to antigen binding.

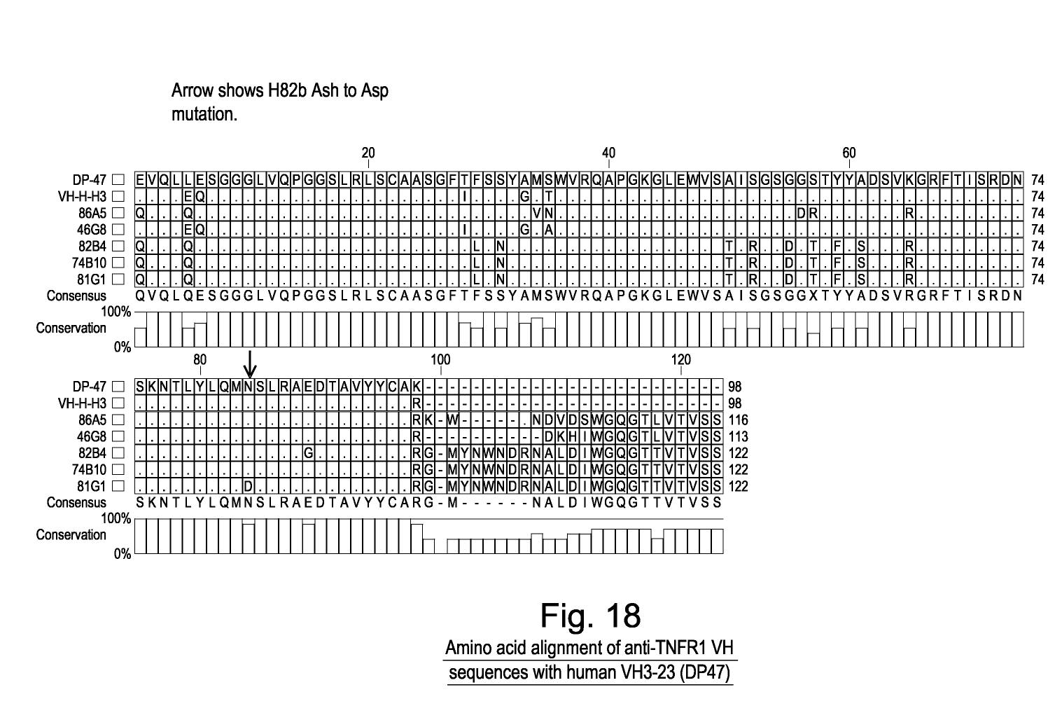

Several anti-TNFR1 VH are described in Example 8 and two of these were taken forward for affinity maturation using standard strategies (Antibody Engineering, Edited by Benny Lo, chapter 8, p 319-359, 2004). One candidate (81G1) was identified following affinity maturation that had a different profile in ELISA relative to other TNFR1 VH from the same lineage (FIG. 17). In this ELISA, several anti-TNFR1 VH (46H6, 74B10, 82B4 and 46G8) bound to TNFR1 as expected but also recognised human TRAIL and human Fas proteins. In addition, an antibody with specificity for KLH (86A5) also bound to human TRAIL and human Fas proteins, as well as human TNFR1. Rather than indicating that these VH are non-specific, the ELISA is demonstrating that the human TRAIL, Fas and TNFR1 preparations contain trace amounts of protein A, present as a result of protein purification processes. VH of the human VH3 family will bind to protein A in these samples and consequently give binding in ELISA (FIG. 19). However, the ELISA also identified a VH with unique binding properties, 81G1 that recognised only human TNFR1 despite also being a VH3 family member. Analysis of the amino acid sequences of the different VH antibodies identified a single amino acid change in 81G1 at Kabat position H82b (Asn to Asp) that abolished protein A binding (FIG. 18), note that this is the only amino acid difference between 81G1 and 74B10. This amino acid change was introduced during the affinity maturation process and corresponds to one of the core binding residues for protein A (Graille M et al, PNAS, 2000; 97(10), 5399-5404). Although this mutation abolished protein A binding for 81G1, binding to TNFR1 was unaffected indicating that the Asn82bArg amino acid change had no effect on VH functionality, in particular, the levels of binding of 81G1 to TNFR1 as shown in FIG. 17 are at a comparable level to that observed for clones not having this mutation (46H6, 74B10, 82B4, 46G8 and 86A5). In addition, affinity of 81G1 for TNFR1 was determined by BIAcore analysis and shown to be similar to that observed for 74B10 (100 nM and 76 nM respectively). VH expression yields were similarly unaffected, with 81G1 successfully purified from a shake flask culture at a yield of 7.5 mg/litre vs 6 mg/litre for its sibling 74B10.

The inventors have identified a specific amino acid change at Kabat position H82b that not only abolishes binding to protein A, but has the added advantage of maintaining functionality. Therefore the identification of clone 81G1 provides for the generation of a VH3 scaffold and libraries of VH antibodies derived therefrom. Libraries having the Asn82bArg amino acid feature would lack any protein A binding capability and would be a useful tool for working with Fc fusion proteins with no concerns about trace amounts of protein A in samples.

Scaffold sequences
Seq ID No. 1: VH-H-3 amino acid sequence
EVQLEQSGGGLVQPGGSLRLSCAASGEIFSSYGMTWVRQAPGKGLEWVS
AISGSGGSTYYADSVKGRFTISRDNSKNTLYLQMNSLRAEDTAVYYCAR
Seq ID No. 2: VH3-93 amino acid sequence
QVQLQESGGGLVQPGGSLRLSCAASGFTFSSYAVSWVRQAPGKGLEWVS
AISGSGDRTYYADSVRGRFTISRDNSKNTLYLQMNSLRAEDTAVYYCAR
Seq ID No. 3: 81G1 amino acid sequence
QVQLQESGGGLVQPGGSLRLSCAASGFTLSNYAMSWVRQAPGKGLEWVS
TIRGSDGTTFYSDSVRGRFTISRDNSKNTLYLQMDSLRAEDTAVYYCAR
Seq ID No. 4: VH-H-3 nucleic acid sequence
GAGGTGCAGCTGGAGCAGTCTGGAGGAGGCTTGGTCCAGCCTGGGGGGT
CCCTGAGACTCTCCTGTGCAGCCTCTGGATTCATCTTTAGCAGCTATGG
CATGACCTGGGTCCGCCAGGCTCCAGGGAAGGGGCTGGAGTGGGTCTCA
GCTATCAGTGGTAGTGGTGGTAGCACATACTACGCAGACTCCGTGAAGG
GCCGATTCACCATCTCCAGAGACAATTCCAAGAACACGCTGTATCTGCA
AATGAACAGCCTGAGAGCCGAGGACACGGCCGTGTATTACTGTGCGAGA
Seq ID No. 5: VH3-93 nucleic acid sequence
CAGGTGCAGCTCCAGGAGTCTGGAGGAGGCTTGGTACAGCCTGGGGGGT
CCCTGAGACTCTCCTGTGCGGCCTCTGGATTCACCTTTAGCAGCTATGC
CGTGAGCTGGGTCCGCCAGGCTCCAGGGAAGGGGCTGGAGTGGGTCTCA
GCTATTAGTGGTAGTGGTGATAGGACATACTACGCAGACTCCGTGAGGG
GCCGGTTCACCATCTCCAGAGACAATTCCAAGAACACGCTGTATCTGCA
AATGAACAGCCTGAGAGCCGAGGACACGGCCGTGTATTACTGTGCAAGA
Seq ID No. 6: 81G1 nucleic acid sequence
CAGGTGCAGCTCCAGGAGTCTGGGGGAGGCTTGGTACAGCCTGGGGGGT
CCCTGAGACTCTCCTGTGCAGCTTCCGGGTTCACCCTTAGCAACTATGC
CATGAGCTGGGTCCGCCAGGCTCCAGGGAAGGGGCTGGAGTGGGTCTCA
ACTATTCGTGGCAGTGATGGTACCACATTCTACTCAGACTCTGTGAGGG
GCCGATTCACCATCTCCAGAGACAATTCCAAGAACACGCTGTATCTGCA
AATGGACAGCCTGAGAGCCGAGGACACGGCCGTGTATTACTGTGCGAGA
G

1. A human VH scaffold capable of producing a VH domain expression library comprising at least 70% soluble clones.

2. A human VH scaffold according to claim 1 having at least 80%, 90%, 95% or 98% amino acid sequence identity with the sequences according to Seq ID No. 1, Seq ID No. 2 or Seq ID No. 3.

3. A human VH scaffold or fragment thereof of claim 1 according to Seq ID No 1.

4. A human VH scaffold or fragment thereof of claim 1 according to Seq ID No 2.

5. A human VH scaffold or fragment thereof of claim 1 according to Seq ID No 3.

6. A human VH scaffold or fragment thereof according to claims 1-5 which is derived from human germline gene V3-23.

7. A human VH scaffold according to claims 1-6 further comprising a CDR3 region.

8. A method for identifying a VH scaffold comprising the steps of:
a) Obtaining a human VH domain expression library
b) Screening the library of step a) against a generic ligand
c) Identifying VH domains which bind the generic ligand and expressing in E. coli
d) Detecting soluble VH domains expressed in step c)
e) Determining the sequence of soluble VH domains to obtain a VH scaffold sequence

9. The method of claim 8 wherein the generic ligand is protein A.

10. The method of claim 8 or 9 wherein the VH domain library is expressed using ribosome display.

11. The method of claims 8-10 wherein the VH scaffold is according to claims 1-6.

12. A method of constructing a VH domain expression library comprising the steps of;
a) Assembling the scaffolds according to claims 1-6 with a plurality of CDR3 nucleic acid sequences to obtain a VH domain repertoire
b) Expressing the VH domain repertoire to produce a VH domain library and selecting for functional VH domains against target antigen.

13. The method of claim 12 wherein the scaffolds are defined according to Seq ID No. 1, Seq ID No. 2, Seq ID No. 3, Seq ID No. 4, Seq ID No. 5 or Seq ID No. 6.

14. The method of claim 13 wherein the scaffolds have at least 80%, 90%, 95% or 98% amino acid sequence identity with the sequences according to Seq ID No. 1, Seq ID No. 2 or Seq ID No. 3.

15. The method of claims 12-14 wherein the selected VH domains are sequenced and/or expressed in a host cell.

16. The method of claims 12-15 comprising the step of CDR3 mutagenesis followed by further rounds of screening.

17. A human VH domain expression library comprising a scaffold according to claims 1-7.

18. A human VH domain expression library according to claim 17 comprising at least 10⁹ unique VH domains.

19. A human VH domain expression library according to claims 17-18 comprising a CDR3 domain derived from a human naïve repertoire.

20. A human VH domain expression library according to claims 17-19 expressed on the surface of a filamentous bacteriophage.

21. An isolated human VH domain or fragment thereof comprising a scaffold as defined in claims 1-7.

22. A pharmaceutical composition comprising a human VH domain according to claim 21 in an effective amount for binding to a target antigen and a pharmaceutically acceptable excipient.
